(19) United States
(12) Patent Application Publication
(10) Pub. No.: US 2026/0207271 A1
(43) Pub. Date: Jul. 23, 2026

(54) SYSTEMS AND METHODS FOR INSERTING A ROBOTIC ASSEMBLY INTO AN INTERNAL BODY CAVITY

(71) Applicant: VICARIOUS SURGICAL INC., Waltham, MA (US)

(72) Inventors: Spencer K. Howe, Scituate, MA (US); Tabitha A. Solomon, Arlington, MA (US); Maxim Antinori, Arlington, MA (US); John Foy, Kailua, HI (US)

(21) Appl. No.: 19/140,244

(22) PCT Filed: Dec. 20, 2023
(86) PCT No.: PCT/US2023/085062

§ 371 Date: Jun. 17, 2025

Related U.S. Application Data
(60) Provisional application No. 63/433,972, filed on Dec. 20, 2022.

(51) Int. Cl. A61B 34/30; A61B 17/34; A61B 34/00; A61B 90/00

(52) U.S. Cl. CPC A61B 34/30; A61B 17/3421; A61B 34/25; A61B 90/37; A61B 2034/302; A61B 2034/306

(57) ABSTRACT

Systems and methods for inserting a robotic arm assembly into an internal body cavity are provided. The system inserts a robotic arm assembly through a trocar into an interior cavity of a subject. The system determines that a first articulated joint of a robotic arm exits the trocar and reaches a first position in the interior cavity. The first position indicates that the first articulated joint is free to rotate relative to the trocar. The system allows a hand controller to articulate the first articulated joint within a first volume. The system determines that a second articulated joint of the robotic arm exits the trocar and reaches a second position in the interior cavity. The second position indicates that the second articulated joint is free to rotate relative to the trocar. The system allows the hand controller to articulate the second articulated joint within a second volume.

### CROSS-REFERENCE TO RELATED APPLICATIONS

This application claims the benefit of U.S. Provisional Patent Application No. 63/433,972, filed Dec. 20, 2022, the entire contents of which are incorporated by reference herein in their entirety.

### BACKGROUND OF THE DISCLOSURE

Surgical robotic systems permit a user (also described herein as an “operator” or a “user”) to perform an operation using robotically-controlled instruments to perform tasks and functions during a procedure. However, conventional surgical robotic systems are limited by straight robot arm insertion techniques. If there are obstructions within an abdominal cavity during straight robot arm insertion, the straight robotic arm cannot be easily guided around these obstructions, and additional trocar ports must be used to approach a surgical target.

Alternatively, additional time must be used to clear these obstructions with laparoscopic tools before the robotic instruments can have the freedom to maneuver as required within the abdominal space.

### SUMMARY

A surgical robotic system is presented. The surgical robotic system includes a camera assembly, a robotic arm assembly having a first robotic arm and a second robotic arm, hand controllers graspable by a user of the surgical robotic system to control the first and the second robot arms and the camera assembly, and a trocar. Each of the first and second robotic arms has a plurality of articulated joints. The surgical robotic system also includes a memory storing one or more instructions, a processor configured to or programmed to read the one or more instructions stored in the memory. The processor is operationally coupled to the robotic arm assembly, the hand controllers and the camera assembly. The processor is configured to enter an insertion mode allowing the user to insert the camera assembly and robotic arm assembly through the trocar into an interior cavity of a subject. The processor is further configured to determine that the first robotic arm is inserted in the trocar. The processor is further configured to determine that a first articulated joint of the plurality of articulated joints of the first robotic arm exits the trocar and reaches a first articulated joint inserted position in the interior cavity. The first articulated joint inserted position indicates that the first articulated joint is free to rotate relative to the trocar. The processor is further configured to allow a first hand controller of the hand controllers to articulate the first articulated joint within a first volume upon determination that the first articulated joint reaches the first articulated joint inserted position. The processor is further configured to determine that a second articulated joint of the plurality of articulated joints of the first robotic arm exits the trocar and reaches a second articulated joint inserted position in the interior cavity. The second articulated joint inserted position indicates that the second articulated joint is free to rotate relative to the trocar. The processor is further configured to allow the first hand controller to articulate the second articulated joint within a second volume upon determination that the second articulated joint reaches the second articulated joint inserted position in the interior cavity.

A method for inserting a robotic assembly of a surgical robotic system through a trocar into an interior cavity of a subject is presented. The robotic assembly includes a robotic arm assembly. The method includes inserting a first robotic arm of the robotic arm assembly through the trocar, the first robotic arm having a plurality of articulated joints. The method further includes determining that a first articulated joint of the plurality of articulated joints exits the trocar and reaches a first articulated joint inserted position in the interior cavity. The first articulated joint inserted position indicates that the first articulated joint is free to rotate relative to the trocar. The method further includes enabling, via a first hand controller of the surgical robotic system, articulation of the first articulated joint within a first volume in the interior cavity such that the first articulated joint is able to be articulated by the first hand controller within the first volume. The method further includes determining that a second articulated joint of the plurality of articulated joints exits the trocar and reaches a second articulated joint inserted position in the interior cavity. The second articulated joint inserted position indicates that the second articulated joint is free to rotate relative to the trocar. The method further includes enabling, via the first hand controller, articulation of the second articulated joint within a second volume in the interior cavity such that the second articulated joint is able to be articulated by the first hand controller within the second volume.

### BRIEF DESCRIPTION OF THE DRAWINGS

These and other features and advantages of the present invention will be more fully understood by reference to the following detailed description in conjunction with the attached drawings in which like reference numerals refer to like elements throughout the different views. The drawings illustrate principals of the invention and, although not to scale, show relative dimensions.

FIG. 1 is a diagram illustrating an example surgical robotic system in accordance with some embodiments.

FIG. 2A is an example perspective view of a patient cart including a robotic support system coupled to a robotic subsystem of the surgical robotic system in accordance with some embodiments.

FIG. 2B is an example perspective view of an example operator console of a surgical robotic system of the present disclosure in accordance with some embodiments.

FIG. 3A is a diagram illustrating an example side view of a surgical robotic system performing a surgery within an internal cavity of a subject in accordance with some embodiments.

FIG. 3B is a diagram illustrating an example top view of the surgical robotic system performing the surgery within the internal cavity of the subject of FIG. 3A in accordance with some embodiments.

FIG. 4A is an example perspective view of a single robotic arm subsystem in accordance with some embodiments.

FIG. 4B is an example perspective side view of a single robotic arm of the single robotic arm subsystem of FIG. 4A in accordance with some embodiments.

FIG. 5 is an example perspective front view of a camera assembly and a robotic arm assembly in accordance with some embodiments.

FIG. 6A is an example perspective view of a left hand controller for use in an operator console of a surgical robotic system in accordance with some embodiments.

FIG. 6B is an example perspective view of a right hand controller for use in an operator console of a surgical robotic system in accordance with some embodiments.

FIG. 6C is a perspective view of a left hand controller for use in connection with an operator console of a surgical robotic system in accordance with some embodiments.

FIG. 6D is a perspective view of a right hand controller for use in connection with an operator console of a surgical robotic system in accordance with some embodiments.

FIG. 6E is a perspective view of a left hand controller for use in connection with an operator console of a surgical robotic system in accordance with some embodiments.

FIG. 6F is a perspective view of a right hand controller for use in connection with an operator console of a surgical robotic system in accordance with some embodiments.

FIG. 7 is an example graphical user interface of a robot pose view including a frustum view of a cavity of a patient and a pair of robotic arms of the surgical robotic system, a robot pose view, and a camera view in accordance with some embodiments.

FIG. 8 is a flowchart illustrating steps for inserting a robotic assembly carried out by the surgical robotic system in accordance with some embodiments.

FIGS. 9A-9D illustrate an insertion of a camera assembly through a trocar in accordance with some embodiments.

FIG. 10 illustrates an example robotic arm assembly of FIG. 4B in a simplified segmented form.

FIGS. 11A-11G illustrate an articulated robotic arm insertion of a first robotic arm through a trocar in accordance with some embodiments.

FIGS. 12A-12G illustrate an articulated robotic arm insertion of a second robotic arm through the trocar of FIG. 12 in accordance with some embodiments.

FIGS. 13A and 13B illustrate an articulated robotic arm insertion of a robotic arm while another robotic arm is partially inserted.

FIG. 14 depicts a graphical user interface showing an articulated robotic arm insertion an interior cavity.

FIG. 15 depicts a computing module in more detail in accordance with some embodiments.

FIG. 16 depicts one example of a robot support system (RSS) comprising axes and translating positioning elements about a cavity of a subject in accordance with some embodiments.

FIGS. 17A-17D show one example of various axes of the RSS, including a side view of an insertion axis (FIG. 17A), a side view of a roll axis (FIG. 17B), a side view of a pitch axis (FIG. 17C), and a top view of a yaw axis (FIG. 17D) in accordance with some embodiments.

FIG. 18 depicts a flowchart illustrating steps for repositioning a portion of the robotic assembly to avoid an obstruction in accordance with some embodiments.

### DETAILED DESCRIPTION

During robotic arm insertion through a trocar into a cavity, for example an abdominal cavity, there can be sensitive tissue and/or obstructions that need to be avoided. Conventional insertion techniques have a limited ability to clear the sensitive tissue and/or obstructions.

Laparoscopic techniques or other conventional techniques often need to clear the sensitive tissue and/or obstructions before the insertion instruments or of robotic arms.

Articulated robotic arm insertion as taught herein is a technique that allows a surgical robotic system to fully or partially insert a robotic arm of the robotic arm assembly into a cavity, for example an abdominal space, while avoiding and/or clearing potential obstructions or sensitive tissue. The articulated robotic arm insertion process taught herein enables users (e.g., surgeons) to articulate or otherwise move joints of a robotic arm once the joint clears a terminal end of a trocar. This allows the user to adjust a portion, for example, a hand portion or a forearm portion or an upper arm portion of the robotic arm one joint at a time as it clears the terminal end of the trocar to clear an obstruction, for example, sensitive tissue during the robotic arm insertion process. The articulated robotic arm insertion process taught herein allows the user to precisely control the robotic arm positions as each joint of the robotic arm clears a terminal end of the trocar.

The articulated robotic arm insertion process taught herein provides users an increasing radius of dexterity during robotic arm insertion through the trocar by leveraging articulated joints (e.g., a wrist joint, an elbow joint, and a shoulder joint) of each robotic arm to allow maneuverability around an obstruction or sensitive tissue. This increased mobility during the robotic assembly insertion process allows users to guide the inserted portions (e.g., full arms or partial arms) of each or both of robotic arms to corresponding target positions through a single trocar port by allowing the user to guide the inserted portions of the robotic arms around an obstruction. This saves valuable time that would otherwise be spent clearing obstructions before inserting the robotic arms, or setting up additional trocar ports to approach the target from other locations and orientations.

The articulated robotic arm insertion process taught herein is also able to alter a yaw or a pitch or both of an inserted trocar relative to an insertion axis of a robot support system (RSS). In this manner two additional degrees of freedom are available to the user during the robotic assembly insertion process to navigate past or around sensitive tissue or an obstruction or both. As described in more detail below one or more volumes within a cavity can be defined based on articulating segments of a robotic arm. The ability to alter the yaw or the pitch or both of the inserted trocar relative to the insertion axis of the RSS allows the user to reposition the plunge position as well as the volume within the interior cavity to facilitate insertion of the robotic arms past or around sensitive tissue or an obstruction or both.

In some embodiments, beginning with insertion of the camera assembly, a user can control the movement of the camera assembly through the trocar and into a cavity, for example, the abdominal cavity. Following this, the user can sequentially move each robotic arm of the robotic arm assembly through the trocar, past the camera assembly and around an obstruction. The user is able to control the camera orientation and plunge position at all times, so that the user can observe the progress of the robotic arm insertion. The sequential structure of the articulated robotic arm insertion taught herein can focus on a single robotic arm at a time. This can allow precise focus on maneuverability of each robotic arm, and reduce cognitive load for users as the users can limit their attention on the camera, or one of the two robotic arms during the insertion sequence. The sequential insertion process can also have the advantage of supporting smaller trocars and thus smaller incisions can be made in a patient, thus reducing the trauma experienced by the patient. It should be understood that the camera assembly and the robotic arm assembly, or robotic arms of the robotic arm assembly can be fully or partially inserted in any order or in a specific order. For example, a robotic arm can be followed by the camera assembly and then followed by another robotic arm. A robotic arm can be inserted during the insertion process of the camera assembly (e.g., the camera assembly is partially inserted) or during the insertion process of another robotic arm (e.g., the robotic arm is partially inserted).

In some embodiments, an articulated robotic arm insertion process as taught herein allows a surgical robotic system to track an articulated robotic arm insertion progress during an articulated robotic arm insertion process and determine when each articulated joint is clear of the terminal end of the trocar. In some embodiments, in order to determine sufficient clearance of a robotic arm joint past the terminal end of the trocar, a point can be chosen far enough inside the trocar according to a physical radius of the articulated joint so that the articulated joint can move and no longer impact the trocar, as described below in detail. In some embodiments, a surgical robotic system of the present disclosure can guide a user through the articulated robotic arm insertion process using visual onscreen prompts, as a user controls each robotic component using a combination of hand controllers and foot pedals, as described below in detail.

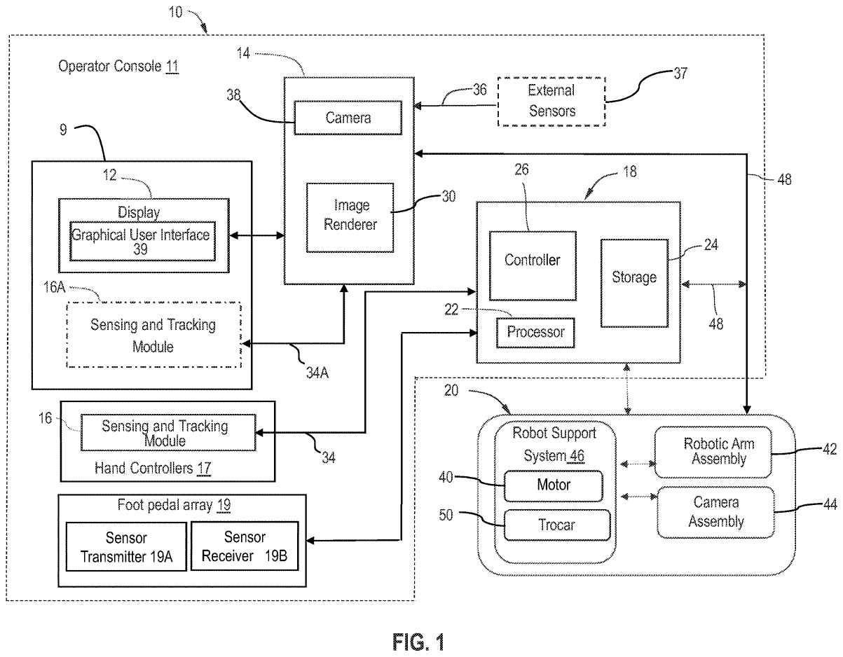

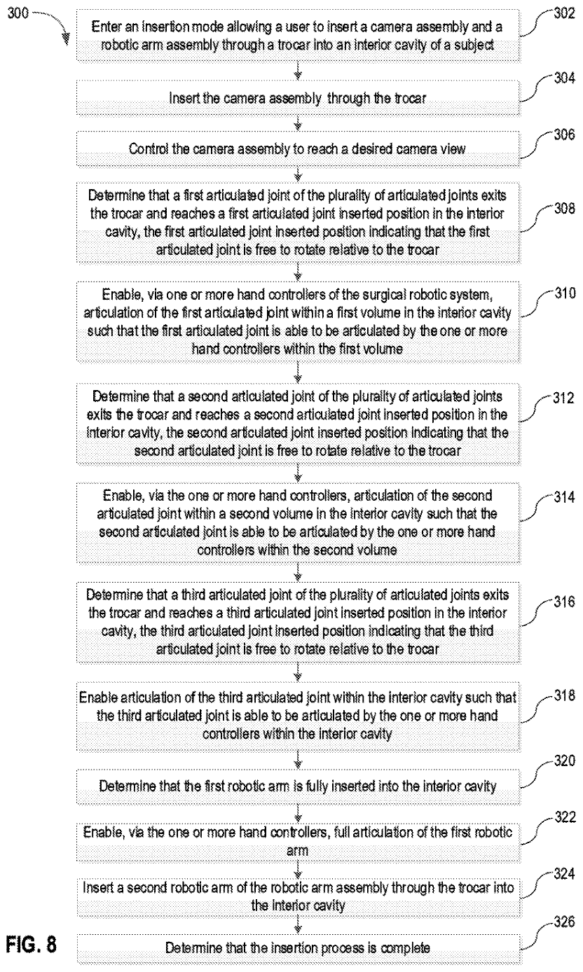

Prior to providing additional specific description of the articulated robotic arm insertion with respect to FIGS. 8-15 a surgical robotic system in which some embodiments could be employed is described below with respect to FIGS. 1-7.

While various embodiments have been taught and described herein, it will be clear to those skilled in the art that such embodiments are provided by way of example only. Numerous variations, changes, and substitutions can occur to those skilled in the art without departing from the invention. It can be understood that various alternatives to the embodiments taught herein can be employed.

As used in the specification and claims, the singular form “a,” “an,” and “the” include plural references unless the context clearly dictates otherwise. It will be further understood that the terms “comprises” and/or “comprising,” or “include” and/or “including,” when used in this specification, specify the presence of stated features, integers, steps, operations, elements, and/or components, but do not preclude the presence or addition of one or more other features, integers, steps, operations, elements, components, and/or groups thereof. As used herein, the term “and/or” includes any and all combinations of one or more of the associated listed items.

Unless specifically stated or obvious from context, as used herein, the term “about” is understood as within a range of normal tolerance in the art, for example within 2 standard deviations of the mean. “About” can be understood as within 10%, 9%, 8%, 7%, 6%, 5%, 4%, 3%, 2%, 1%, 0.5%, 0.1%, 0.05%, or 0.01% of the stated value. Unless otherwise clear from the context, all numerical values provided herein are modified by the term “about.”

Although some example embodiments can be described herein or in documents incorporated by reference as employing a plurality of units to perform example processes, it is understood that example processes can also be performed by one or a plurality of modules. Additionally, it is understood that the term controller/controller can refer to a hardware device that includes a memory and a processor and is specifically programmed to execute the processes described herein in accordance with some embodiments. In some embodiments, the memory is configured to store the modules and the processor is specifically configured to execute said modules to perform one or more processes which are described further below. In some embodiments, multiple different controllers or controllers or multiple different types of controllers or controllers can be employed in performing one or more processes. In some embodiments, different controllers or controllers can be implemented in different portions of a surgical robotic systems.

### Surgical Robotic Systems

Some embodiments can be employed with a surgical robotic system. A system for robotic surgery can include a robotic subsystem. The robotic subsystem includes at least a portion, which can also be referred to herein as a robotic assembly herein, that can be inserted into a patient via a trocar through a single incision point or site. The portion inserted into the patient via a trocar is small enough to be deployed in vivo at the surgical site and is sufficiently maneuverable when inserted to be able to move within the body to perform various surgical procedures at multiple different points or sites. The portion inserted into the body that performs functional tasks can be referred to as a surgical robotic module, a surgical robotic module or a robotic assembly herein. The surgical robotic module can include multiple different submodules or parts that can be inserted into the trocar separately. The surgical robotic module, surgical robotic module or robotic assembly can include multiple separate robotic arms that are deployable within the patient along different or separate axes. These multiple separate robotic arms can be collectively referred to as a robotic arm assembly herein. Further, a surgical camera assembly can also be deployed along a separate axis. The surgical robotic module, surgical robotic module, or robotic assembly can also include the surgical camera assembly. Thus, the surgical robotic module, or robotic assembly employs multiple different components, such as a pair of robotic arms and a surgical or robotic camera assembly, each of which are deployable along different axes and are separately manipulatable, maneuverable, and movable. The robotic arms and the camera assembly that are disposable along separate and manipulatable axes is referred to herein as the Split Arm (SA) architecture. The SA architecture is designed to simplify and increase efficiency of the insertion of robotic surgical instruments through a single trocar at a single insertion site, while concomitantly assisting with deployment of the surgical instruments into a surgical ready state as well as the subsequent removal of the surgical instruments through the trocar. By way of example, a surgical instrument can be inserted through the trocar to access and perform an operation in vivo in the abdominal cavity of a patient. In some embodiments, various surgical instruments can be used or employed, including but not limited to robotic surgical instruments, as well as other surgical instruments known in the art.

The systems, devices, and methods taught herein can be incorporated into and/or used with a robotic surgical device and associated system taught for example in U.S. Pat. No. 10,285,765 and in PCT patent application Serial No. PCT/US2020/39203, and/or with the camera assembly and system taught in United States Publication No. 2019/0076199, and/or the systems and methods of exchanging surgical tools in an implantable surgical robotic system taught in PCT patent application Serial No. PCT/US2021/058820, where the content and teachings of all of the foregoing patents, patent applications and publications are incorporated herein by reference herein in their entirety. The surgical robotic module that forms part of the present invention can form part of a surgical robotic system that includes a user workstation that includes appropriate sensors and displays, and a robot support system (RSS) for interacting with and supporting the robotic subsystem of the present invention in some embodiments. The robotic subsystem includes a motor and a surgical robotic module that includes one or more robotic arms and one or more camera assemblies in some embodiments. The robotic arms and camera assembly can form part of a single support axis robotic system, can form part of the split arm (SA) architecture robotic system, or can have another arrangement. The robot support system can provide multiple degrees of freedom such that the robotic module can be maneuvered within the patient into a single position or multiple different positions. In one embodiment, the robot support system can be directly mounted to a surgical table or to the floor or ceiling within an operating room. In another embodiment, the mounting is achieved by various fastening means, including but not limited to, clamps, screws, or a combination thereof. In other embodiments, the structure can be free standing. The robot support system can mount a motor assembly that is coupled to the surgical robotic module, which includes the robotic arm assembly and the camera assembly. The motor assembly can include gears, motors, drivetrains, electronics, and the like, for powering the components of the surgical robotic module.

The robotic arm assembly and the camera assembly are capable of multiple degrees of freedom of movement. According to some embodiments, when the robotic arm assembly and the camera assembly are inserted into a patient through the trocar, they are capable of movement in at least the axial, yaw, pitch, and roll directions. The robotic arms of the robotic arm assembly are designed to incorporate and employ a multi-degree of freedom of movement robotic arm with an end effector mounted at a distal end thereof that corresponds to a wrist area or joint of the user. In other embodiments, the working end (e.g., the end effector end) of the robotic arm is designed to incorporate and use or employ other robotic surgical instruments, such as for example the surgical instruments set forth in U.S. Pub. No. 2018/0221102, the entire contents of which are herein incorporated by reference.

Like numerical identifiers are used throughout the figures to refer to the same elements.

FIG. 1 is a schematic illustration of an example surgical robotic system 10 in which aspects of the present disclosure can be employed in accordance with some embodiments of the present disclosure. The surgical robotic system 10 includes an operator console 11 and a robotic subsystem 20 in accordance with some embodiments.

The operator console 11 includes a display 12, an image computing module 14, which can be a three-dimensional (3D) computing module, hand controllers 17 having a sensing and tracking module 16, and a computing module 18. Additionally, the operator console 11 can include a foot pedal array 19 including a plurality of pedals. The image computing module 14 can include a graphical user interface 39. The graphical user interface 39, the controller 26 or the image renderer 30, or both, can render one or more images or one or more graphical user interface elements on the graphical user interface 39. For example, a pillar box associated with a mode of operating the surgical robotic system 10, or any of the various components of the surgical robotic system 10, can be rendered on the graphical user interface 39. Also live video footage captured by a camera assembly 44 can also be rendered by the controller 26 or the image renderer 30 on the graphical user interface 39.

The operator console 11 can include a visualization system 9 that includes a display 12 which can be any selected type of display for displaying information, images or video generated by the image computing module 14, the computing module 18, and/or the robotic subsystem 20. The display 12 can include or form part of, for example, a head-mounted display (HMD), an augmented reality (AR) display (e.g., an AR display, or AR glasses in combination with a screen or display), a screen or a display, a two-dimensional (2D) screen or display, a three-dimensional (3D) screen or display, and the like. The display 12 can also include an optional sensing and tracking module 16A. In some embodiments, the display 12 can include an image display for outputting an image from a camera assembly 44 of the robotic subsystem 20.

The hand controllers 17 are configured to sense a movement of the operator's hands and/or arms to manipulate the surgical robotic system 10. The hand controllers 17 can include the sensing and tracking module 16, circuity, and/or other hardware. The sensing and tracking module 16 can include one or more sensors or detectors that sense movements of the operator's hands. In some embodiments, the one or more sensors or detectors that sense movements of the operator's hands are disposed in the hand controllers 17 that are grasped by or engaged by hands of the operator. In some embodiments, the one or more sensors or detectors that sense movements of the operator's hands are coupled to the hands and/or arms of the operator. For example, the sensors of the sensing and tracking module 16 can be coupled to a region of the hand and/or the arm, such as the fingers, the wrist region, the elbow region, and/or the shoulder region. Additional sensors can also be coupled to a head and/or neck region of the operator in some embodiments. In some embodiments, the sensing and tracking module 16 can be external and coupled to the hand controllers 17 via electricity components and/or mounting hardware. In some embodiments, the optional sensor and tracking module 16A can sense and track movement of one or more of an operator's head, of at least a portion of an operator's head, an operator's eyes or an operator's neck based, at least in part, on imaging of the operator in addition to or instead of by a sensor or sensors attached to the operator's body.

In some embodiments, the sensing and tracking module 16 can employ sensors coupled to the torso of the operator or any other body part. In some embodiments, the sensing and tracking module 16 can employ in addition to the sensors an Inertial Momentum Unit (IMU) having for example an accelerometer, gyroscope, magnetometer, and a motion processor. The addition of a magnetometer allows for reduction in sensor drift about a vertical axis. In some embodiments, the sensing and tracking module 16 also include sensors placed in surgical material such as gloves, surgical scrubs, or a surgical gown. The sensors can be reusable or disposable. In some embodiments, sensors can be disposed external of the operator, such as at fixed locations in a room, such as an operating room. The external sensors 37 can generate external data 36 that can be processed by the computing module 18 and hence employed by the surgical robotic system 10.

The sensors generate position and/or orientation data indicative of the position and/or orientation of the operator's hands and/or arms. The sensing and tracking modules 16 and/or 16A can be utilized to control movement (e.g., changing a position and/or an orientation) of the camera assembly 44 and robotic arm assembly 42 of the robotic subsystem 20. The tracking and position data 34 generated by the sensing and tracking module 16 can be conveyed to the computing module 18 for processing by at least one processor 22.

The computing module 18 can determine or calculate, from the tracking and position data 34 and 34A, the position and/or orientation of the operator's hands or arms, and in some embodiments of the operator's head as well, and convey the tracking and position data 34 and 34A to the robotic subsystem 20. The tracking and position data 34, 34A can be processed by the processor 22 and can be stored for example in the storage 24. The tracking and position data 34 and 34A can also be used by the controller 26, which in response can generate control signals for controlling movement of the robotic arm assembly 42 and/or the camera assembly 44. For example, the controller 26 can change a position and/or an orientation of at least a portion of the camera assembly 44, of at least a portion of the robotic arm assembly 42, or both. In some embodiments, the controller 26 can also adjust the pan and tilt of the camera assembly 44 to follow the movement of the operator's head.

The robotic subsystem 20 can include a robot support system (RSS) 46 having a motor 40 and a trocar 50 or trocar mount, the robotic arm assembly 42, and the camera assembly 44. The robotic arm assembly 42 and the camera assembly 44 can form part of a single support axis robot system, such as that taught and described in U.S. Pat. No. 10,285,765, or can form part of a split arm (SA) architecture robot system, such as that taught and described in PCT Patent Application No. PCT/US2020/039203, both of which are incorporated herein by reference in their entirety.

The robotic subsystem 20 can employ multiple different robotic arms that are deployable along different or separate axes. In some embodiments, the camera assembly 44, which can employ multiple different camera elements, can also be deployed along a common separate axis. Thus, the surgical robotic system 10 can employ multiple different components, such as a pair of separate robotic arms and the camera assembly 44, which are deployable along different axes. In some embodiments, the robotic arm assembly 42 and the camera assembly 44 are separately manipulatable, maneuverable, and movable. The robotic subsystem 20, which includes the robotic arm assembly 42 and the camera assembly 44, is disposable along separate manipulatable axes, and is referred to herein as an SA architecture. The SA architecture is designed to simplify and increase efficiency of the insertion of robotic surgical instruments through a single trocar at a single insertion point or site, while concomitantly assisting with deployment of the surgical instruments into a surgical ready state, as well as the subsequent removal of the surgical instruments through the trocar 50 as further described below.

The RSS 46 can include the motor 40 and the trocar 50 or a trocar mount. The RSS 46 can further include a support member that supports the motor 40 coupled to a distal end thereof. The motor 40 in turn can be coupled to the camera assembly 44 and to each of the robotic arm assembly 42. The support member can be configured and controlled to move linearly, or in any other selected direction or orientation, one or more components of the robotic subsystem 20. In some embodiments, the RSS 46 can be free standing. In some embodiments, the RSS 46 can include the motor 40 that is coupled to the robotic subsystem 20 at one end and to an adjustable support member or element at an opposed end.

The motor 40 can receive the control signals generated by the controller 26. The motor 40 can include gears, one or more motors, drivetrains, electronics, and the like, for powering and driving the robotic arm assembly 42 and the cameras assembly 44 separately or together. The motor 40 can also provide mechanical power, electrical power, mechanical communication, and electrical communication to the robotic arm assembly 42, the camera assembly 44, and/or other components of the RSS 46 and robotic subsystem 20. The motor 40 can be controlled by the computing module 18. The motor 40 can thus generate signals for controlling one or more motors that in turn can control and drive the robotic arm assembly 42, including for example the position and orientation of each robot joint of each robotic arm, as well as the camera assembly 44. The motor 40 can further provide for a translational or linear degree of freedom that is first utilized to insert and remove each component of the robotic subsystem 20 through the trocar 50. The motor 40 can also be employed to adjust the inserted depth of each robotic arm of the robotic arm assembly 42 when inserted into the patient 100 through the trocar 50.

The trocar 50 is a medical device that can be made up of an awl (which can be a metal or plastic sharpened or non-bladed tip), a cannula (essentially a hollow tube), and a seal in some embodiments. The trocar 50 can be used to place at least a portion of the robotic subsystem 20 in an interior cavity of a subject (e.g., a patient) and can withdraw gas and/or fluid from a body cavity. The robotic subsystem 20 can be inserted through the trocar 50 to access and perform an operation in vivo in a body cavity of a patient. In some embodiments, the robotic subsystem 20 can be supported, at least in part, by the trocar 50 or a trocar mount with multiple degrees of freedom such that the robotic arm assembly 42 and the camera assembly 44 can be maneuvered within the patient into a single position or multiple different positions. In some embodiments, the robotic arm assembly 42 and camera assembly 44 can be moved with respect to the trocar 50 or a trocar mount with multiple different degrees of freedom such that the robotic arm assembly 42 and the camera assembly 44 can be maneuvered within the patient into a single position or multiple different positions.

In some embodiments, the RSS 46 can further include an optional controller for processing input data from one or more of the system components (e.g., the display 12, the sensing and tracking module 16, the robotic arm assembly 42, the camera assembly 44, and the like), and for generating control signals in response thereto. The motor 40 can also include a storage element for storing data in some embodiments.

The robotic arm assembly 42 can be controlled to follow the scaled-down movement or motion of the operator's arms and/or hands as sensed by the associated sensors in some embodiments and in some modes of operation. The robotic arm assembly 42 include a first robotic arm including a first end effector at distal end of the first robotic arm, and a second robotic arm including a second end effector disposed at a distal end of the second robotic arm. In some embodiments, the robotic arm assembly 42 can have portions or regions that can be associated with movements associated with the shoulder, elbow, and wrist joints as well as the fingers of the operator. For example, the robotic elbow joint can follow the position and orientation of the human elbow, and the robotic wrist joint can follow the position and orientation of the human wrist. The robotic arm assembly 42 can also have associated therewith end regions that can terminate in end-effectors that follow the movement of one or more fingers of the operator in some embodiments, such as for example the index finger as the user pinches together the index finger and thumb. In some embodiments, while the robotic arm assembly 42 can follow movement of the arms of the operator in some modes of control while a virtual chest of the robotic assembly can remain stationary (e.g., in an instrument control mode). In some embodiments, the position and orientation of the torso of the operator are subtracted from the position and orientation of the operator's arms and/or hands. This subtraction allows the operator to move his or her torso without the robotic arms moving. Further disclosure control of movement of individual arms of a robotic assembly is provided in International Patent Application Publications WO 2022/094000 A1 and WO 2021/231402 A1, each of which is incorporated by reference herein in its entirety.

The camera assembly 44 is configured to provide the operator with image data 48, such as for example a live video feed of an operation or surgical site, as well as enable the operator to actuate and control the cameras forming part of the camera assembly 44. In some embodiments, the camera assembly 44 can include one or more cameras (e.g., a pair of cameras), the optical axes of which are axially spaced apart by a selected distance, known as the inter-camera distance, to provide a stereoscopic view or image of the surgical site. In some embodiments, the operator can control the movement of the cameras via movement of the hands via sensors coupled to the hands of the operator or via hand controllers 17 grasped or held by hands of the operator, thus enabling the operator to obtain a desired view of an operation site in an intuitive and natural manner. In some embodiments, the operator can additionally control the movement of the camera via movement of the operator's head. The camera assembly 44 is movable in multiple directions, including for example in yaw, pitch and roll directions relative to a direction of view. In some embodiments, the components of the stereoscopic cameras can be configured to provide a user experience that feels natural and comfortable. In some embodiments, the interaxial distance between the cameras can be modified to adjust the depth of the operation site perceived by the operator.

The image or video data 48 generated by the camera assembly 44 can be displayed on the display 12. In embodiments in which the display 12 includes an HMD, the display can include the built-in sensing and tracking module 16A that obtains raw orientation data for the yaw, pitch and roll directions of the HMD as well as positional data in Cartesian space (x, y, z) of the HMD. In some embodiments, positional and orientation data regarding an operator's head can be provided via a separate head-tracking module. In some embodiments, the sensing and tracking module 16A can be used to provide supplementary position and orientation tracking data of the display in lieu of or in addition to the built-in tracking system of the HMD. In some embodiments, no head tracking of the operator is used or employed. In some embodiments, images of the operator can be used by the sensing and tracking module 16A for tracking at least a portion of the operator's head.

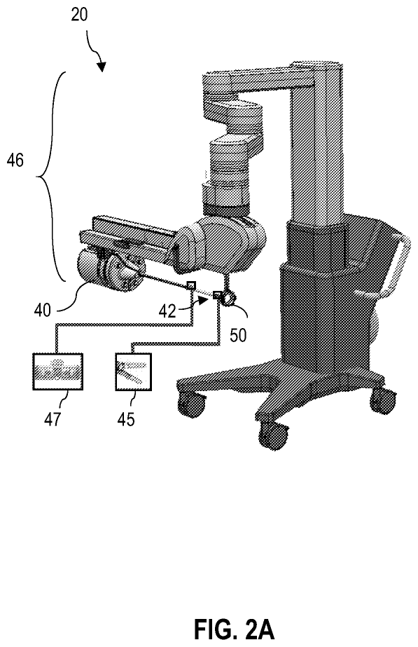

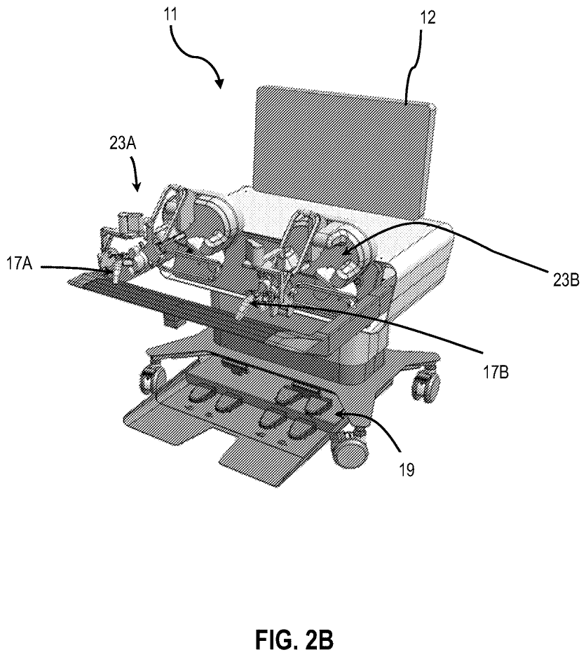

FIG. 2A depicts an example robotic assembly 20, which is also referred to herein as a robotic subsystem, of a surgical robotic system 10 incorporated into or mounted onto a mobile patient cart in accordance with some embodiments. In some embodiments, the robotic subsystem 20 includes the RSS 46, which, in turn includes the motor 40, the robotic arm assembly 42 having end-effectors 45, the camera assembly 44 having one or more cameras 47, and can also include the trocar 50 or a trocar mount.

FIG. 2B depicts an example of an operator console 11 of the surgical robotic system 10 of the present disclosure in accordance with some embodiments. The operator console 11 includes a display 12, hand controllers 17, and also includes one or more additional controllers, such as a foot pedal array 19 for control of the robotic arm assembly 42, for control of the camera assembly 44, and for control of other aspects of the system.

FIG. 2B also depicts the left hand controller subsystem 23A and the right hand controller subsystem 23B of the operator console. The left hand controller subsystem 23A includes and supports the left hand controller 17A and the right hand controller subsystem 23B includes and supports the right hand controller 17B. In some embodiments, the left hand controller subsystem 23A can releasably connect to or engage the left hand controller 17A, and right hand controller subsystem 23B can releasably connect to or engage the right hand controller 17A. In some embodiments, the connections can be both physical and electronic so that the left hand controller subsystem 23A and the right hand controller subsystem 23B can receive signals from the left hand controller 17A and the right hand controller 17B, respectively, including signals that convey inputs received from a user selection on a button or touch input device of the left hand controller 17A or the right hand controller 17B.

Each of the left hand controller subsystem 23A and the right hand controller subsystem 23B can include components that enable a range of motion of the respective left hand controller 17A and right hand controller 17B, so that the left hand controller 17A and right hand controller 17B can be translated or displaced in three dimensions and can additionally move in the roll, pitch, and yaw directions. Additionally, each of the left hand controller subsystem 23A and the right hand controller subsystem 23B can register movement of the respective left hand controller 17A and right hand controller 17B in each of the forgoing directions and can send a signal providing such movement information to the processor 22 (as shown in FIG. 1) of the surgical robotic system 10.

In some embodiments, each of the left hand controller subsystem 23A and the right hand controller subsystem 23B can be configured to receive and connect to or engage different hand controllers (not shown). For example, hand controllers with different configurations of buttons and touch input devices can be provided. Additionally, hand controllers with a different shape can be provided. The hand controllers can be selected for compatibility with a particular surgical robotic system or a particular surgical robotic procedure or selected based upon preference of an operator with respect to the buttons and input devices or with respect to the shape of the hand controller in order to provide greater comfort and ease for the operator.

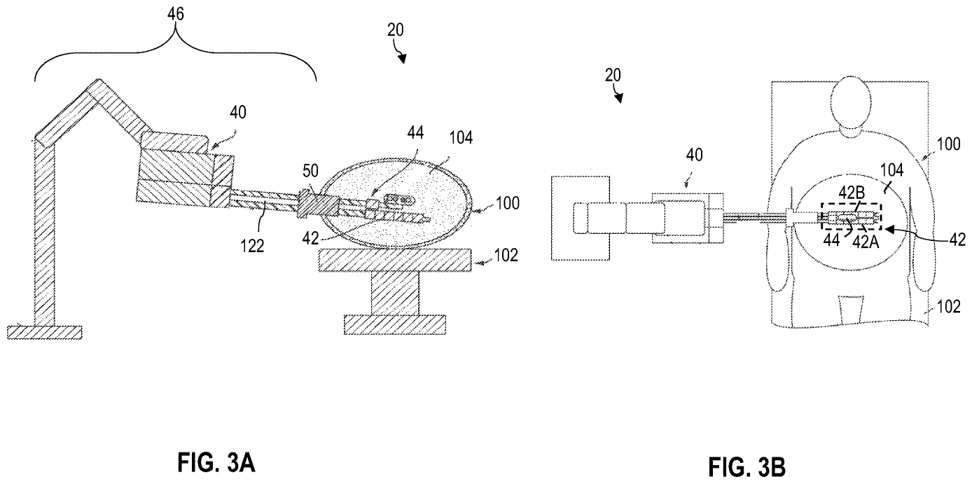

FIG. 3A schematically depicts a side view of the surgical robotic system 10 performing a surgery within an internal cavity 104 of a subject 100 in accordance with some embodiments and for some surgical procedures. FIG. 3B schematically depicts a top view of the surgical robotic system 10 performing the surgery within the internal cavity 104 of the subject 100. The subject 100 (e.g., a patient) is placed on an operation table 102 (e.g., a surgical table 102). In some embodiments, and for some surgical procedures, an incision is made in the patient 100 to gain access to the internal cavity 104. The trocar 50 is then inserted into the patient 100 at a selected location to provide access to the internal cavity 104 or operation site. The RSS 46 can then be maneuvered into position over the patient 100 and the trocar 50. In some embodiments, the RSS 46 includes a trocar mount that attaches to the trocar 50. The camera assembly 44 and the robotic arm assembly 42 can be coupled to the motor 40 and inserted individually and/or sequentially into the patient 100 through the trocar 50 and hence into the internal cavity 104 of the patient 100. Although the camera assembly 44 and the robotic arm assembly 42 can include some portions that remain external to the subject's body in use, references to insertion of the robotic arm assembly 42 and/or the camera assembly 44 into an internal cavity of a subject and disposing the robotic arm assembly 42 and/or the camera assembly 44 in the internal cavity of the subject are referring to the portions of the robotic arm assembly 42 and the camera assembly 44 that are intended to be in the internal cavity of the subject during use. The sequential insertion method has the advantage of supporting smaller trocars and thus smaller incisions can be made in the patient 100, thus reducing the trauma experienced by the patient 100. In some embodiments, the camera assembly 44 and the robotic arm assembly 42 can be inserted in any order or in a specific order. In some embodiments, the camera assembly 44 can be followed by a first robotic arm 42A of the robotic arm assembly 42 and then followed by a second robotic arm 42B of the robotic arm assembly 42 all of which can be inserted into the trocar 50 and hence into the internal cavity 104. Once inserted into the patient 100, the RSS 46 can move the robotic arm assembly 42 and the camera assembly 44 to an operation site manually or automatically controlled by the operator console 11.

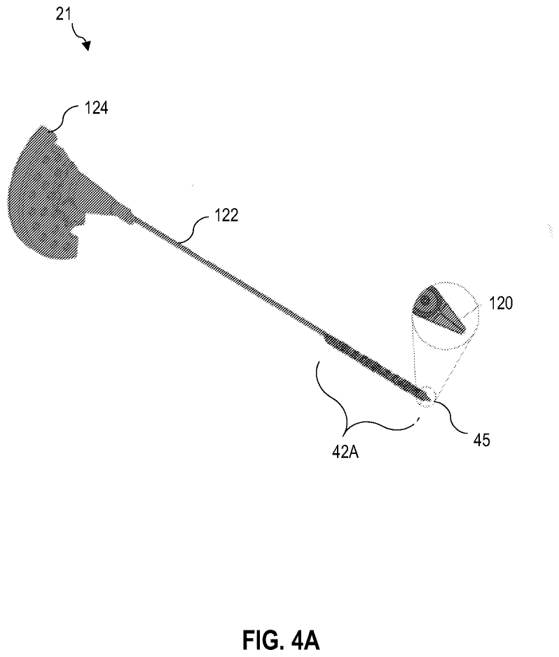

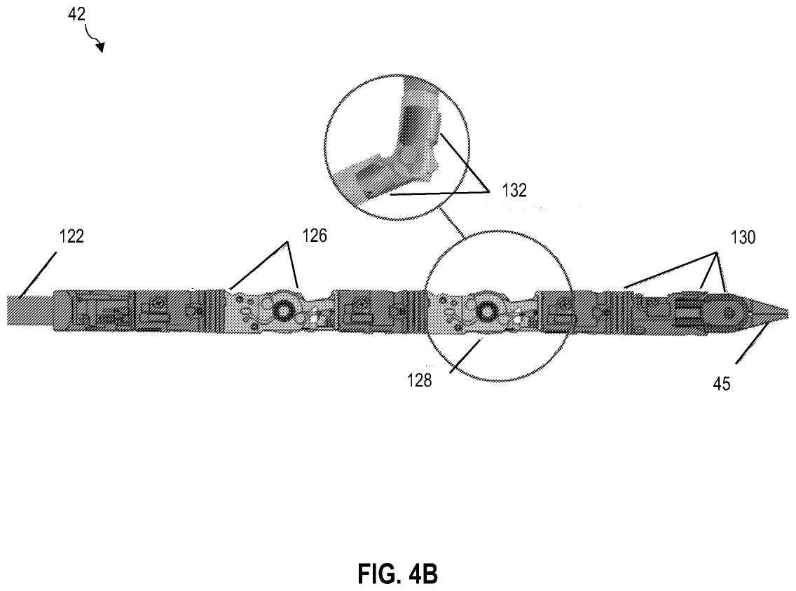

Further disclosure regarding control of movement of individual arms of a robotic arm assembly is provided in International Patent Application Publications WO 2022/094000 A1 and WO 2021/231402 A1, each of which is incorporated by reference herein in its entirety. FIG. 4A is a perspective view of a robotic arm subassembly 21 in accordance with some embodiments. The robotic arm subassembly 21 includes a robotic arm assembly 42, the end-effector 45 having an instrument tip 120 (e.g., monopolar scissors, needle driver/holder, bipolar grasper, or any other appropriate tool), a support tube 122 supporting the robotic arm assembly 42. A distal end of the support tube 122 is coupled to the robotic arm assembly 42, and a proximal end of the support tube 122 is coupled to a housing 124 of the motor 40 (as shown in FIG. 2A). At least a portion of the support tube 122 can be external to the internal cavity 104 (as shown in FIGS. 3A and 3B). At least a portion of the support tube 122 can be inserted into the internal cavity 104 (as shown in FIGS. 3A and 3B). The support tube 122, can be inserted through the trocar (e.g., along a longitudinal axis of the trocar). The support tube 122 may be configured to provide mechanical and structural support to the trocar and the positioning elements and wrist elements. The support tube may be configured to provide electrical power or electrical control signals (e.g., via electrical cables) to the positioning elements, wrist elements, the camera, for performing cautery, and/or the like including combinations and/or multiples thereof. According to one or more embodiments described herein, the support tube may be configured to provide a separate coaxial cable or the like for video captured by the camera.

FIG. 4B is a side view of the robotic arm assembly 42. The robotic arm assembly 42 includes a shoulder joint 126 forming a virtual shoulder, an elbow joint 128 having position sensors 132 (e.g., capacitive proximity sensors) and forming a virtual elbow, a wrist joint 130 forming a virtual wrist, and the end-effector 45 in accordance with some embodiments. The shoulder joint 126, the elbow joint 128, the wrist joint 130 can include a series of hinge and rotary joints to provide each arm with positionable, seven degrees of freedom, along with one additional grasping degree of freedom for the end-effector 45 in some embodiments. In some embodiments, the surgical robotic system 10 as a whole has nine degrees of freedom.

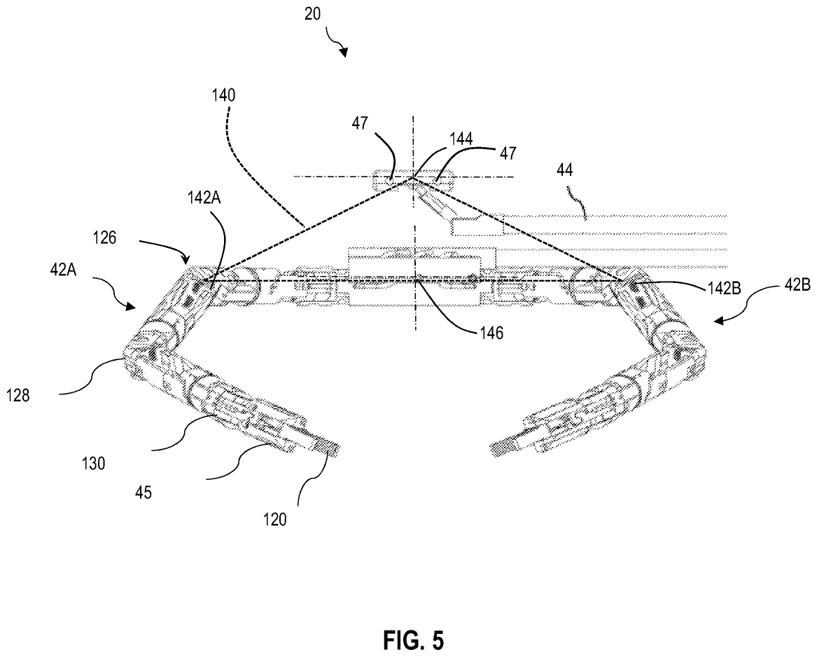

FIG. 5 illustrates a perspective front view of a portion of the robotic assembly 20 configured for insertion into an internal body cavity of a patient. The robotic assembly 20 includes a first robotic arm 42A and a second robotic arm 42B. The two robotic arms 42A and 42B can define, or at least partially define, a virtual chest 140 of the robotic assembly 20 in some embodiments. In some embodiments, the virtual chest 140 (depicted as a triangle with dotted lines) can be defined by a chest plane extending between a first pivot point 142A of a most proximal joint of the first robotic arm 42A (e.g., a shoulder joint 126), a second pivot point 142B of a most proximal joint of the second robotic arm 42B, and a camera imaging center point 144 of the camera(s) 47. A pivot center 146 of the virtual chest 140 lies in the middle of the virtual chest 140.

In some embodiments, sensors in one or both of the first robotic arm 42A and the second robotic arm 42B can be used by the surgical robotic system 10 to determine a change in location in three-dimensional space of at least a portion of each or both of the robotic arms 42A and 42B. In some embodiments, sensors in one or both of the first robotic arm 42A and second robotic arm 42B can be used by the surgical robotic system 10 to determine a location in three-dimensional space of at least a portion of one robotic arm relative to a location in three-dimensional space of at least a portion of the other robotic arm.

In some embodiments, the camera assembly 44 is configured to obtain images from which the surgical robotic system 10 can determine relative locations in three-dimensional space. For example, the camera assembly 44 can include multiple cameras, at least two of which are laterally displaced from each other relative to an imaging axis, and the system can be configured to determine a distance to features within the internal body cavity. Further disclosure regarding a surgical robotic system including camera assembly and associated system for determining a distance to features can be found in International Patent Application Publication No. WO 2021/159409, entitled “System and Method for Determining Depth Perception In Vivo in a Surgical Robotic System,” and published Aug. 12, 2021, which is incorporated by reference herein in its entirety. Information about the distance to features and information regarding optical properties of the cameras can be used by a system to determine relative locations in three-dimensional space.

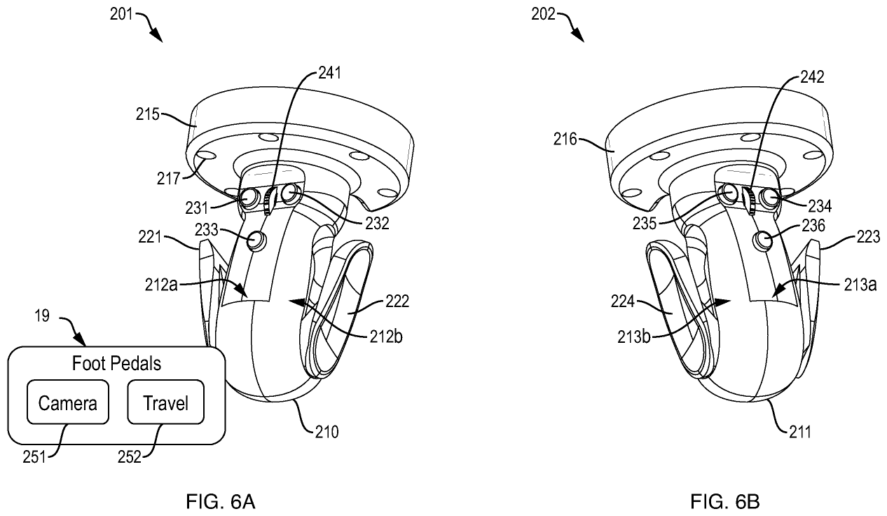

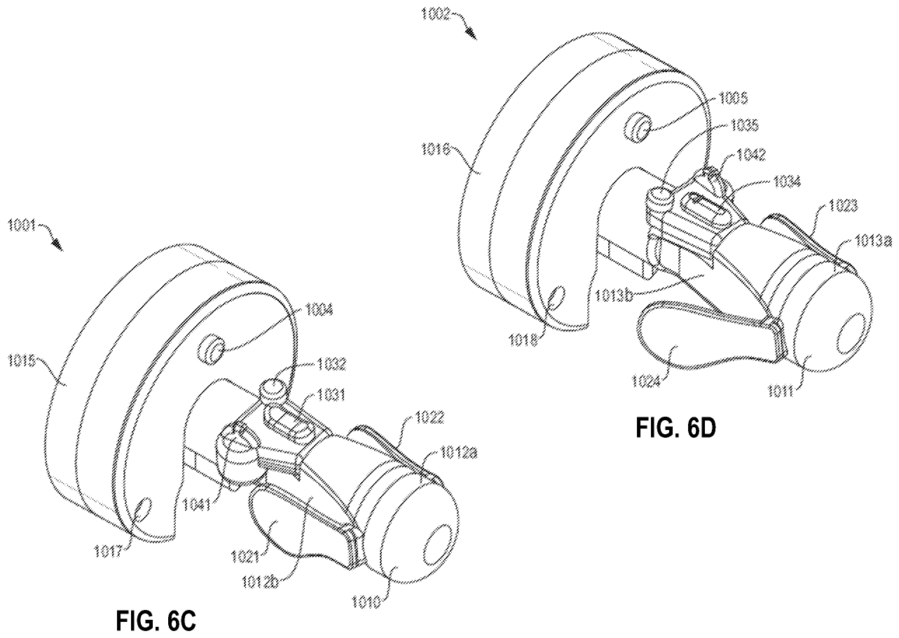

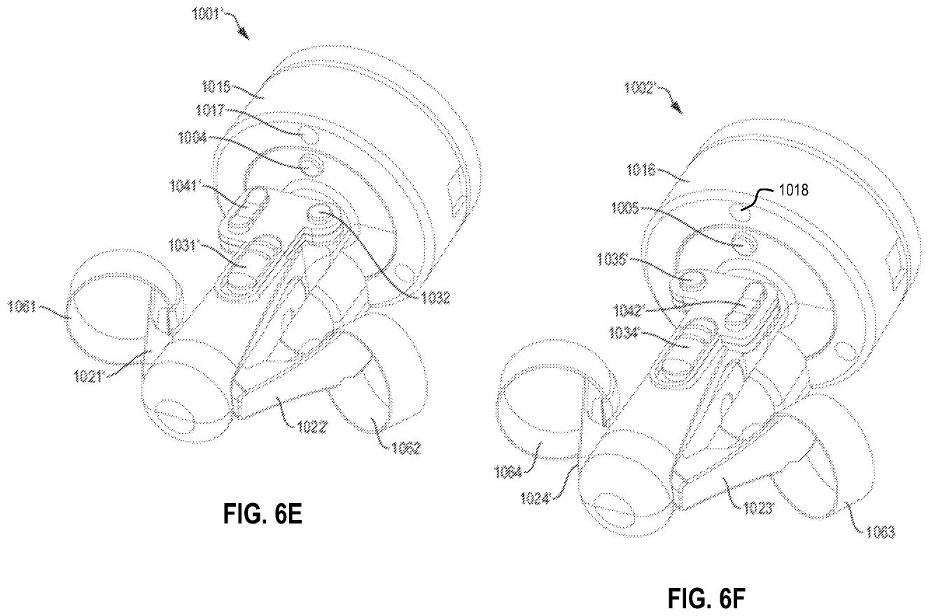

FIG. 6A depicts a left hand controller 201 and FIG. 6B depicts a right hand controller 202 in accordance with some embodiments. The left hand controller 201 and the right hand controller 202 each include a contoured housing 210, 211, respectively. Each contoured housing 210, 211, includes an upper surface 212a, 213a, an inside side surface 212b, 213b adjacent the upper surface, an outside side surface (not visible in these views) facing away from the inside side surface 212b, 213b, and a lower surface (not visible in these views) facing away from the upper surface 212a, 213a.

In some embodiments, each hand controller 201, 202 includes a mounting assembly 215, 216, respectively. The mounting assembly 215, 216 can be used to attach, either directly or indirectly, the respective hand controller 201, 202 to a user console of a surgical robotic system. In some embodiments, the mounting assembly 215 defines holes 217, which can be countersunk holes, configured to receive a screw or bolt to connect the left hand controller 201 to a user console.

In some embodiments, such as that depicted in FIGS. 6A and 6B, the hand controller includes two control levers, three buttons, and one touch input device. As will be explained herein, embodiments can feature other combinations of touch input devices, buttons, and levers, or a subset thereof. The embodiment shown as the left hand controller 201 features a first control lever 221 and a second control lever 222. Similarly, right hand controller 202 includes a first control lever 223 and a second control lever 224. In some embodiments, first control lever 221 is engaged with the second control lever 222 via one or more gears (not shown) so that a user depressing the first control lever 221 causes a reciprocal movement in the second control lever 222 and vice versa. In another embodiment, the first control lever 221 and second control lever 222 can be configured to operate independently. In embodiments employing reciprocal movement of the first and second control lever, a hand controller can employ only one signal indicating a deflection of the first lever and the second lever. In embodiments in which the first control lever and second control lever operate independently, a hand controller can employ a first signal indicating a deflection of the first control lever and a second signal indicating a deflection of the second control lever.

In some embodiments, the first control lever 221, 223 and the second control lever 222, 224 can be contoured to receive a thumb and/or finger of a user. In some embodiments, the first control lever 221, 223 extends from or extends beyond the outside side surface of the respective contoured housing 210, 211 the second control lever 222, 224 extends from or extends beyond the inside side surface 212b, 212c of the respective contoured housing. For each hand controller 210, 211, deflection or depression of the first control lever 221, 223, and the second control lever 222, 224, is configured to produce a signal that the surgical robotic system uses as an input to control a tool or an instrument tip (e.g., opening/closing an aperture of graspers/jaws of an instrument tip) at a distal end of a robotic arm of the surgical robotic system. For example, depressing first control lever and the second control lever can change an angle of jaws of a grasper at a distal end of the respective robotic arm. In some embodiments, end effectors, tools or instruments are used to pull tissue apart, drive a needle driver, grab an item (e.g., a mesh, suture, needle) or pick up such an item in the body cavity when it is dropped, deliver energy via an electrosurgical unit (ESU) (e.g., to cut or to coagulate).

In some embodiments, a housing of a hand controller can be contoured. For example, in FIGS. 6A and 6B, the contoured housing 210, 211 includes a rounded shape. In some embodiments, a housing can be shaped to have a contour to match a contour of at least a portion of a thumb of a user's hand. In some embodiments, the contoured housing 210, 211, the first control lever 221, 223, and the second control lever 222, 224, can each be shaped to comfortably and ergonomically receive a respective hand of a user. In some embodiments, a housing of the hand controller, a lever or levers of a hand controller, buttons of a hand controller and/or one or more touch input devices can have shapes and/or positions on the hand controller for fitting different palm sizes and finger lengths.

Left hand controller 201 also includes a first button 231, a second button 232, and a third button 233. Similarly, right hand controller 202 also includes a first button 234, a second button 235 and a third button 236. As taught herein, each button can provide one or more inputs that can be mapped to a variety of different functions of the surgical robotic device to control the surgical robotic system including a camera assembly and a robotic arm assembly. In an embodiment, input received via the first button 231 of the left hand controller 201 and input received via the first button 234 of the right hand controller 202 can control a clutch feature. For example, by engaging the first button 231, 234 a clutch is activated enabling movement of the respective left hand controller 201 or right hand controller 20, by the operator without causing any movement of a robotic arms assembly (e.g., a first robotic arm, a second robotic arm, and a camera assembly) of the surgical robotic system. When the clutch is activated for a hand controller, movement of the respective right hand controller or left hand controller is not translated to movement of the robotic assembly. In some embodiments, an operator engaging a hand controller input (e.g., tapping or pressing a button) activates the clutch and the operator engaging again (e.g., tapping or pressing the button again) turns off the clutch or exits a clutch mode. In some embodiments, an operator engaging a hand controller input (e.g., tapping or pressing a button and holding the button) activates the clutch and the clutch stays active for as long as the input is active and exits the clutch when the when the operator is no longer engaging the hand controller input (e.g., releasing the button). Activating the clutch or entering the clutch mode for a hand controller enables the operator to reposition the respective hand controller (e.g., re-position the left controller 201 within the range of motion of the left hand controller 201 and/or re-position the right hand controller 202 within a range of motion of the right hand controller 202) without causing movement of the robotic arms assembly itself.

The second button 232 of the left hand controller 201 can provide an input that controls a pivot function of the surgical robotic device. An operator engaging (e.g., pressing and holding) the second button 232 of the left hand controller 201 can engage a pivot function or a pivot mode that reorients the robotic arms assembly chest to center the camera on the midpoint between the instrument tips. The pivot function can be activated with a brief tap or held down to continuously track the instrument tips as they move, in accordance with some embodiments.

The second button 235 of the right hand controller 202 can provide input for entering a menu mode in which a menu is displayed on the display 12 of the surgical robotic system 10 and exiting a menu mode. The operator can activate a menu mode by pressing the second button 235 a first time and disengage the menu function by pressing the second button 235 a second time. The operator can be able to select options within the menu by navigating the menu using the left hand controller and/or the right hand controller when the menu mode is engaged. For example, the first touch input device 242 of the right hand controller 202 can be used to navigate the menu and to select a menu item in some embodiments. While in a menu mode, movement of the robotic in response to movement of the left hand controller 201 or the right hand controller 202 can be suspended. The third button 233 of the left hand controller and the third button of the right hand controller can provide an input that engages or disengages an instrument control mode of the surgical robotic system in some embodiments. A movement of at least one of the one or more hand controllers when in the instrument mode causes a corresponding movement in a corresponding robotic arm of the robotic assembly.

The left hand controller 201 further includes a touch input device 241. Similarly, the right hand controller 202 further includes a touch input device 242. In an embodiment, the touch input device 241, 242 can be a scroll wheel, as shown in FIGS. 6A and 6B. Other touch input devices that can be employed include, but are not limited to, rocker buttons, joy sticks, pointing sticks, touch pads, track balls, trackpoint nubs, etc.

The touch input device 241, 242 can be able to receive input through several different forms of engagement by the operator. For example, where the touch input device 241, 242 is a scroll wheel, the operator can be able to push or click the first touch input device 241, 242, scroll the first touch input device 241, 242 backward or forward, or both.

In some embodiments, scrolling the first touch input device 241 of the left hand controller 241 forward can activate a zoom in function to magnify a view provided by the camera assembly of the surgical robotic system and displayed to the operator, and scrolling backward with first touch input device 241 can provide a zoom out function to reduce the view provided by the camera assembly of the surgical robotic device and displayed to the operator, or vice versa. In embodiments, the zoom function can be mechanical or digital. In some embodiments, the zoom function can be mechanical in part and digital in part (e.g., a mechanical zoom over one zoom range, and a mechanical zoom plus a digital zoom over another zoom range).

In some embodiments, clicking or depressing first touch input device 241 can engage a scan mode of the surgical robotic system. When in a scan mode, a movement of at least one of the left hand controller 201 or the right hand controller 202 causes a corresponding change in an orientation of a camera assembly of the robotic arms assembly without changing a position or orientation of either robotic arm of the surgical robotic system. In another embodiment, pressing and holding the first touch input device 241 can activate the scan mode and releasing the first touch input device 241 can end the scan mode of the surgical robotic system. In some embodiments, releasing the scan mode returns the camera to the orientation it was in upon entering scan mode. In some embodiments, a function can be provided for locking the orientation upon exiting the scan mode (e.g., to change the “horizon” line).

In some embodiments, when in a menu mode and a left elbow menu item is selected, the first touch input device 241 of the left hand controller 201 can be used for selection of a direction and degree of left elbow bias. As used herein, elbow bias refers to the extent by which the virtual elbow of the robotic arm is above or below a neutral or default position.

In some embodiments, when in a menu mode, an operator can be able to select options within the menu by navigating the menu using the left hand controller and/or the right hand controller. For example, when in the menu mode, the touch input device 242 (e.g., scroll wheel) of the right hand controller provides a set of inputs for traversing a displayed menu and selecting an item in a displayed menu. For example, by scrolling forward on touch input device 242 the operator can move up the menu and by scrolling backwards with touch input device 242 the user can move down the menu, or vice versa. In an embodiment, by clicking first touch input device 242 the operator can make a selection within a menu.

In some embodiments, the touch input device 242 of the right hand controller 202 can be used to control right elbow bias when a right elbow bias menu item has been selected.

Functions of various buttons and the touch input device described above with respect to the left hand controller above can instead be assigned to the right hand controller, and functions of various buttons and the touch input device described above with respect to the right hand controller can instead be assigned to the left hand controller in some embodiments.

FIG. 6A also shows a schematic depiction of a foot pedal array 19 having a first foot pedal 251 and second foot pedal 252 for receiving operator input. As shown in FIG. 6A, in some embodiments the first foot pedal 251 engages a camera control mode, also described herein as a view control mode, an image framing control mode, or a camera framing control mode of the surgical robotic system and the second foot pedal 252 engages a travel control mode of the surgical robotic system.

In some embodiment, when the camera control mode is activated e.g., using the foot pedal 251, movement of the left hand controller 201 and/or the right hand controller 202 by the operator can provide input that is interpreted by the system to control a movement of and an orientation of a camera assembly of the surgical robotic system while keeping positions of instrument tips of robotic arms of the robotic arms assembly constant.

In some embodiments, when the travel control mode is activated e.g., using the foot pedal 252, the left hand controller 201 and the right hand controller 202 can be used to move the robotic arm assembly of the surgical robotic system in a manner in which distal tips of the robotic arms direct or lead movement of a chest of the robotic arms assembly through an internal body cavity. In the travel control mode, a position and orientation of the camera assembly, of the chest, or of both is automatically adjusted to maintain the view of the camera assembly directed at the tips (e.g., at a point between a tip or tips of a distal end of the first robotic arm and a tip or tips of a distal end of the second robotic arm). This can be described as the camera assembly being pinned to the chest of the robotic arms assembly and automatically following the tips. Further detail regarding the travel control mode is provided below.

FIGS. 6C and 6D depict another embodiment according to the present disclosure featuring a left hand controller 1001 and a right hand controller 1002. The left hand controller 1001 includes a contoured housing 1010, and the right hand controller 1002 includes a contoured housing 1011. Each contoured housing 1010, 1011, includes an upper surface 1012a, 1013a, an inside side surface 1012b, 1013b adjacent the upper surface, an outside side surface (not visible in these views) facing away from the inside side surface 1012b, 1013b, and a lower surface (not visible in these views) facing away from the upper surface 1012a, 1013a.

Each hand controller 1001, 1002 includes a mounting assembly 1015, 1016, respectively. The mounting assembly 1015, 1016 that may be used to attach, either directly or indirectly, each of the respective hand controllers 1001, 1002 to a surgeon console of a surgical robotic system. The mounting assembly 1015 includes an aperture 1017 and the mounting assembly 1016 defines an aperture 1018. The apertures 1017, 1018 may be countersunk apertures, configured to receive a screw or bolt to connect the respective hand controller 1001, 1002 to a surgeon console. The mounting assembly 1015 includes a button 1004 and the mounting assembly 1016 includes a button 1005. The buttons 1004, 1005 provide an input to toggle between insertion and extraction of one or more robotic arms 42A, 42B as well as the camera assembly 44. For example, the button 1004 can be used to insert or extract a first robotic arm 42A and the button 1005 can be used to insert or extract a second robotic arm 42B. In some embodiments, the buttons 1004, 1005 do not actually control insertion or extraction of the camera assembly 44, but allow an operator to enter a mode of insertion or extraction. The actual processes for the camera assembly 44 can be controlled by other user elements.

Each of the left hand controller 1001 and the right hand controller 1002 also includes a first button 1031, 1034, a second button 1032, 1035, a touch input device 1041, 1042 (e.g., a joy stick, or scroll wheel), respectively. In each hand controller 1001, 1002, the first button 1021, 1034, the second button 1032, 1035, and the touch input device 1041, 1042 are disposed on or at an upper surface 1012a, 1013a of the housing 1010, 1011, respectively. In some embodiments, the first button 1021, 1034, the second button 1032, 1035, and the touch input device 1041, 1042 are disposed on or at a portion of the upper surface 1012a, 1013a that projects from the upper surface. For each hand controller 1001, 1002, a lever (not visible in this view) extends from the respective outside side surface (not visible in this view). In some embodiments, a different mechanism may be used for a grasping input on a hand controller. For example, in some embodiments a hand controller may include a least one “pistol trigger” type button that can be pulled back to close and released to open instead of or in addition to a lever or levers.

The left hand controller 1001 includes a first paddle 1021 and a second paddle 1022. Similarly, right hand controller 1002 includes a first paddle 1023 and a second paddle 1024. In some embodiments, first paddle 1021, 1023 is engaged with the second paddle 1022, 1024 of each hand controller 1001, 1002 via one or more gears (not shown) so that a user depressing the first paddle 1021, 1023 causes a reciprocal movement in the second paddle 1022, 1024 and vice versa, respectively. In another embodiment, the first paddle 1021, 1023 and the second paddle 1022, 1024 of each hand controller may be configured to operate independently. In embodiments employing reciprocal movement of the first and second paddles, the hand controller 1001, 1002 may employ some form of a signal or other indicator indicating a deflection of the first paddle 1021, 1023 and the second paddle 1022, 1024. In embodiments in which the first paddle and second paddle operate independently, the hand controller 1001, 1002 may employ a first signal or other indicator indicating a deflection of the first paddle 1021, 1023 and a second signal or other indicator indicating a deflection of the second paddle 1022, 1024.

In some embodiments, the first paddle 1021, 1023 and the second paddle 1022, 1024 may be contoured to receive a thumb and/or finger of a user. In some embodiments, the first paddle 1021, 1023 extends from or extends beyond the outside side surface of the respective contoured housing 1010, 1011 the second paddle 1022, 1024 extends from or extends beyond the inside side surface 1012b, 1013b of the respective contoured housing. For each hand controller 1010, 1011, deflection or depression of the first paddle 1021, 1023, and the second paddle 1022, 1024, is configured to trigger a signal that the surgical robotic system uses as an input to control a tool or an instrument tip (e.g., opening/closing an aperture of graspers/jaws of an instrument tip) at a distal end of a robotic arm of the surgical robotic system. For example, depressing first paddle 1021, 1023 and the second paddle 1022, 1024 may change an angle of jaws of a grasper at a distal end of the respective robotic arm. In some embodiments, end effectors, tools or instruments are used to pull tissue apart, drive a needle driver, grab an item (e.g., a mesh, suture, needle) or pick up such an item in the body cavity when it is dropped, deliver energy via an electrosurgical unit (ESU) (e.g., to cut or to coagulate).

In some embodiments, each of the first paddle 1021, 1023 and the second paddle 1022, 1024 can have a loop to receive a thumb and/or finger of a user, as further described with respect to FIGS. 6E and 6F. In some embodiments, parameters (e.g., length, angle, finger ergonomics, and the like) of each of the first paddle 1021, 1023 and the second paddle 1022, 1024 can be adjusted.

The contoured housing 1010, 1011 may be configured to comfortably and ergonomically mate with a corresponding hand of the operator. The operator may engage with the respective hand controller 1001, 1002 by placing the thumb of the respective hand on the second paddle 1022, 1024, positioning the pointer finger or middle finger of the respective hand on or over the projecting portion of the upper surface 1013a, 1013a on which the first button 1021, 1034, the second button 1032, 1035, and the touch input device 1041, 1042 are disposed, and by positioning at least, the middle finger or ring finger of the respective hand on or over the first paddle 1021, 1024.

Although various example embodiments described herein assign certain functions to certain buttons and to certain touch input devices, one of ordinary skill of the art in view of the present disclosure will appreciate that which functions are ascribed to which buttons and touch input devices may be different in different embodiments. Further, one of ordinary skill of the art in view of the present disclosure will appreciate that additional functions not explicitly described herein may be assigned to some buttons and some touch input devices in some embodiments. In some embodiments, one or more functions may be assigned to a foot pedal of a surgical robotic system that includes one or more hand controllers as described herein.

By way of example, a set of functions that may be controlled by the left hand controller 1001 and the right hand controller 1002 for some embodiments of the present technology will now be described.

For left hand controller 1001, pressing or pressing and holding the first button 1004 may trigger a signal used to engage an insertion or extraction for a left robotic arm assembly and/or a camera assembly of the surgical robotic system. Pressing or pressing and holding the first button 1031 may trigger a signal used to control a clutch function for the left hand controller of the surgical robotic system. Pressing or pressing and holding the second button 1032 may trigger a signal used to engage or disengage a camera control mode of the surgical robotic system. Scrolling the touch input device 1041 forward may activate a zoom in function to magnify a view provided by the camera assembly of the surgical robotic system and displayed to the operator, and scrolling backward with first touch input device 1041 may provide a zoom out function to reduce the view provided by the camera assembly of the surgical robotic device and displayed to the operator, or vice versa. Scrolling the touch input device 1041 may trigger a signal used to select left elbow bias when an elbow bias function is activated using a menu.

For right hand controller 1002, pressing or pressing and holding the first button 1005 may trigger a signal used to engage an insertion or extraction for a right robotic arm assembly and/or a camera assembly of the surgical robotic system. Pressing or pressing and holding the first button 1034 may trigger a signal used to control a clutch function for the right hand controller of the surgical robotic system. Clicking or depressing the second button 1035 may engage a scan mode of the surgical robotic system. When in a scan mode, a movement of at least one of the left hand controller 1001 or the right hand controller 1002 causes a corresponding change in an orientation of a camera assembly of the robotic assembly without changing a position or orientation of either robotic arm of the surgical robotic system. In another embodiment, pressing and holding the second button 1035 may activate the scan mode and releasing the second button 1035 may end the scan mode of the surgical robotic system. In some embodiments, releasing the scan mode returns the camera to the orientation it was in upon entering the scan mode. In some embodiments, a function may be provided for locking the orientation upon exiting the scan mode (e.g., to change the “horizon” line). Scrolling the touch input device 1042 may trigger a signal used to traverse a menu or highlight a portion of the menu when the menu is displayed or a menu mode is active. Pressing the touch input device 1042 may trigger a signal used to select a highlighted portion or of the menu or feature on the menu when the menu is displayed. Scrolling the touch input device 1042 may produce a signal used to select right elbow bias when the elbow bias function is activated using the menu. Scrolling forward on touch input device 1042 may move up the menu and scrolling backwards with touch input device 1042 may move down the menu, or vice versa. Clicking first touch input device 1042 may make a selection within a menu.

FIGS. 6E and 6F depict another embodiment according to the present disclosure featuring a left hand controller 1001′ and a right hand controller 1002′. Compared with the hand controllers 1001, 1002 in FIGS. 6C and 6D, some buttons of the hand controllers 1001′, 1002′ have the same button type but different functions. For example, the second button 1035′ of the right hand controller 1002′ may trigger a signal used to turn on or turn off a menu. Compared with the hand controllers 1001, 1002 in FIGS. 6C and 6D, some buttons of the hand controllers 1001′, 1002′ may have a different button type and/or different functions. For example, touch input device 1041′ for the left hand controller 1001′ may have a three-way switch button type. Switching or holding the touch input device 1041′ to the center may trigger a signal used to engage or disengage a scan mode of the surgical robotic system. Switching the touch input device 1041′ forward may activate a zoom in function to magnify a view provided by the camera assembly of the surgical robotic system and displayed to the operator, and switching backward with first touch input device 1041′ may provide a zoom out function to reduce the view provided by the camera assembly of the surgical robotic device and displayed to the operator, or vice versa. Switching the touch input device 1035′ upward may trigger a signal used to traverse a menu when the menu is displayed or a menu mode is active. Touch input device 1042′ for the right hand controller 1002′ may have a three-way switch button type. Switching the touch input device 1042′ may trigger a signal used to traverse a menu or highlight a portion of the menu when the menu is displayed or a menu mode is active by pressing the touch input device 1035′. Switching forward on touch input device 1042′ may move up the menu and switching backwards with touch input device 1042′ may move down the menu, or vice versa. Clicking first touch input device 1042′ may trigger a signal used to select a highlighted portion or of the menu or feature on the menu when the menu is displayed. In some embodiments, switching the touch input device 1042′ may trigger a signal used to select right elbow bias when the elbow bias function is activated using the menu. Compared with the hand controllers 1001, 1002 in FIGS. 6C and 6D, the hand controllers 1001′, 1002′ may have the first paddles 1021′, 1023′ and second paddles 1022′, 1024′ to couple to finger loops 1061, 1062, 1063, 1064, respectively. Each finger loop can be a Velcro type. In some embodiments (not illustrated), each finger loop can be a hook type. Deflection or depression of the first paddle 1021′, 1023′, and the second paddle 1022′, 1024′, is configured to trigger a signal to control a tool or an instrument tip (e.g., opening/closing an aperture of graspers/jaws of an instrument tip) at a distal end of a robotic arm of the surgical robotic system. For example, depressing first paddle 1021′, 1023′ and the second paddle 1022′, 1024′ may change an angle of jaws of a grasper at a distal end of the respective robotic arm. In some embodiments, end effectors, tools or instruments are used to pull tissue apart, drive a needle driver, grab an item (e.g., a mesh, suture, needle) or pick up such an item in the body cavity when it is dropped, deliver energy via an electrosurgical unit (ESU) (e.g., to cut or to coagulate). Compared with the hand controllers 1001, 1002 in FIGS. 6C and 6D, first buttons 1031′, 1034′ may have a slider button type. Sliding the first button 1031′, 1034′ may trigger a signal used to control a clutch function for the corresponding hand controller of the surgical robotic system.

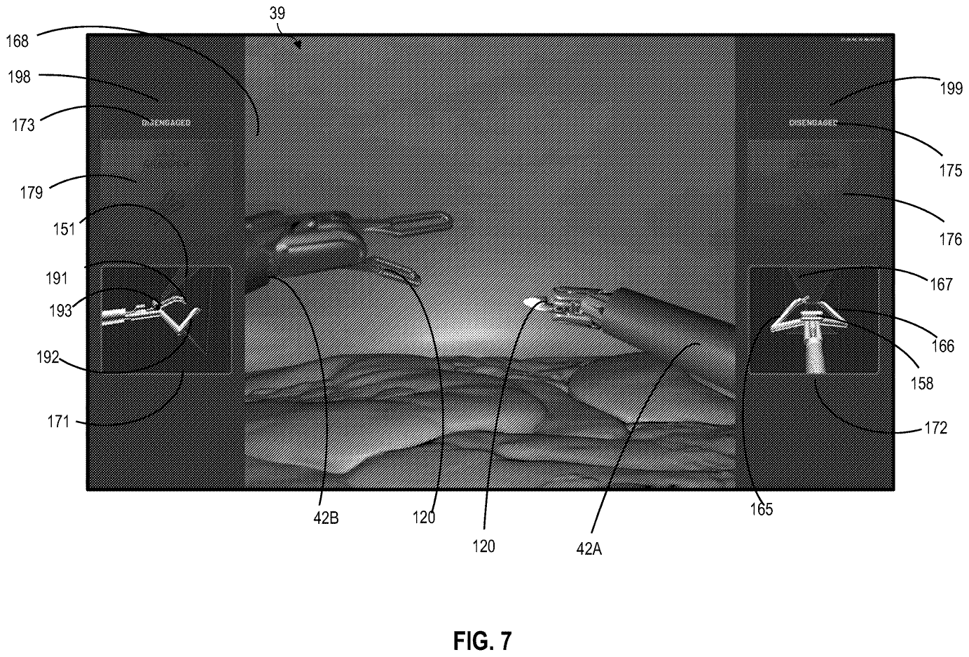

FIG. 7 is a graphical user interface 39 that is formatted to include a left pillar box 198 198 and a right pillar box 199 to the left and right of a live video footage 168, respectively, of a cavity of a patient. The graphical user interface 39 can be overlaid over the live video footage 168. In some embodiments, the live video footage 168 is formatted by the controller 26 to accommodate the left pillar box 198 and the right pillar box 199. In some embodiments, the live video footage 168 can be displayed on display 12, with a predetermined size and location on the display 12, and the left pillar box 198 and the right pillar box 199 can be displayed on either side of the live video footage 168 with a certain size based on the remaining area on the display 12 that is not occupied by the live video footage 168. The graphical user interface 39 includes multiple different graphical user interface elements, which are described below in more detail.

Robotic arms 42B and 42A are also visible in the live video footage. The left pillar box 198 can include a status identifier 173, for example, an engaged or disengaged status identifier associated with an instrument tip 120 of the robotic arm 42B. The “engaged” status identifier 173 indicates that the user's left hand and arm are engaged with the left hand controller 201 and therefore the instrument tip 120 is also engaged. The “disengaged” status identifier 173 indicates that the user's left hand and arm are not engaged with the hand controller 201 and therefore the instrument tip 120 is also disengaged. When the user's left hand and arm are disengaged with the left hand controller 201, the surgical robotic system 10 can be completely disengaged. That is the surgical robotic system 10 can remain on, but it is unresponsive until the user's hands reengage with the hand controllers. The instrument tip 120 can be represented by iconographic symbol 179 that includes a name of the instrument tip 120 to provide confirmation to the user of what type of end effector or instrument tip is currently in use. In FIG. 7, the instrument tip 120 represented by the iconographic symbol 179 is a bipolar grasper. Notably, the present disclosure is not limited to the bipolar grasper or scissor shown in FIG. 7.

Similarly, the right pillar box 199 can include a status identifier 175 associated with an instrument tip 120 of the robotic arm 42A for example, engaged or disengaged status identifier. In some embodiments, based on the status of the end effector, the graphical user interface can also provide a visual representation of the status in addition to text. For example, the end effector iconography can be “grayed out” or made less prominent if it is not disengaged.

The status identifier 175 can be “engaged” thereby indicating that the user's right hand and arm are engaged with the right hand controller 202 and therefore the instrument tip 120 is also engaged. Alternatively, the status identifier 175 can be “disengaged” thereby indicating that the user's right hand and arm are not engaged with the right hand controller 202 and therefore the instrument tip 120 is also disengaged. The instrument tip 120 can be represented by iconographic symbol 176 that includes a name of the instrument tip 120 to provide confirmation to the user of what type of end effector or instrument tip is currently in use. In FIG. 7, the instrument tip 120 represented by iconographic symbol 176 is monopolar scissors. Notably, the present disclosure is not limited to the monopolar scissors shown in FIG. 7.

The left pillar box 198 can also include a robot pose view 171. The robot pose view 171 includes a simulated view of the robotic arms 42B and 42A, the camera assembly 44, and the support arm thereby allowing the user to get a third person view of the robotic arm assembly 42, the camera assembly 44, and the robot support system 46. The simulated view of the robotic arms 42B and 42A represented by a pair of simulated robotic arms 191 and 192. The simulated view of the camera assembly 44 is represented by a simulated camera 193. The robot pose view 171 also includes a simulated camera view associated with a cavity, or a portion of the cavity, of a patient, which is representative of the placement, or location of the pair of robotic arms 151 and 172 relative to a frustum 151. More specifically the camera view can be the field of view of the camera assembly 44 and is equivalent to the frustum 151.

The right pillar box 199 can also include a robot pose view 172 that includes a simulated view of the robotic arms 42B and 42A, the camera assembly 44, the support arm thereby allowing the user to get a third person view of the robotic arm assembly 42, the camera assembly 44, and the support arm. The simulated view of the robotic arms 42B and 42A are a pair of simulated robotic arms 165 and 166. The simulated view of the camera assembly 44 is represented by a simulated camera 193. The robot pose view 172 also includes a simulated camera view associated with a cavity, or a portion of the cavity, of the patient, which is the placement, or location of the pair of robotic arms 165 and 166 relative to a frustum 167. More specifically, the camera view can be the camera's field of view which is the frustum 167. The robot pose view 172 provides elbow height awareness, and situational awareness especially with driving in up facing/lip facing configurations.

Situational awareness can be characterized as a way of understanding certain robotic elements with respect to time and space when the robotic arms 42A and 42B are inside the cavity of the patient. For example, as shown in the robot pose view 171 the elbow of the simulated robotic arm 192 is bent downwards thereby providing the user with the ability to know how the elbow of the actual robotic arm 42A is actually oriented and positioned within the cavity of the patient. It should be noted that because of the positioning of the camera assembly 44 with respect to the robotic arms 42A and 42B, the entire length of the robotic arms 42A and 42B may not be visible in the live video footage 168. As a result, the user may not have visualization of how the robotic arms 42A and 42B are oriented and positioned within the cavity of the patient. The simulated robotic arms 165 and 166, as well as simulated robotic arms 191 and 192 provide the user with the situational awareness of at least the position and orientation of the actual robotic arms 42A and 42B within the cavity of the patient.

There can be two separate views (the robot pose view 171 and the robot pose view 172) from two different viewpoints on each side of the graphical user interface 39 that is rendered on display 12. The robot pose view 171 and the robot pose view 172 automatically update to stay centered on the trocar 50 while maintaining the robotic arms 42A and 42B in view. The robot pose views 171 and 172 also provide the user with spatial awareness.

Spatial awareness can be characterized as the placement or position of the robotic arms 42A and 42B as viewed in robot pose views 171 and 172 relative to other objects in the cavity and the cavity itself. The robot pose views 171 and 172 provide the user with the ability to determine where the actual robotic arms 42A and 42B are located within the cavity by viewing the simulated robotic arms 191 and 192 in the robot pose view 171 and simulated robotic arms 165 and 166 in robot pose view 172. For example, the robot pose view 171 illustrates the position and location of the simulated robotic arms 191 and 192 relative to the frustum 151. The robot pose view 171 depicts the simulated robotic arms 191 and 192 with respect to the frustum 151, from a side view of the support arm and the simulated robotic arms 191 and 192 that are attached to the support arm. This particular robot pose provides the user with the ability to better ascertain proximity to anatomical features within the cavity.

The robot pose view 172 can also provide the user with the ability to better ascertain how close the actual robotic arms 42A and 42B are relative to one another, or how far apart they are from one another. Further still, the robot pose view 172 can also illustrate where the actual robotic arms 42A and 42B might be positioned or located relative to the inside the cavity of the patient that are to the left and right of the robotic arms 42A and 42B, thereby providing the user with a spatial awareness of where the robotic arms 42A and 42B are within the cavity, and where they are relative to anatomical features within the cavity. As noted above, because the full length of the robotic arms 42A and 42B are not visible in the live video footage 168, the simulated robotic arms 165 and 166 can provide the user with the spatial awareness to know how close or far apart the actual robotic arms 42A and 42B are from one another. The view provided by the robot pose view 172 is a view as if the user were looking at a field of the inside of the cavity. The robot pose view 172 provides the user with the spatial awareness to know how close the virtual elbows 128 are relative to one another if the user manipulates the right hand controller 202 and the left hand controller in such a way that the virtual elbows 128 are brought closer together, as well as how close the actual robotic arms 42A and 42B are to one another. For example, as the user manipulates the left hand controller 201 and the right hand controller 202 to straighten the robotic arms 42A and 42B, simulated robotic arms 166 and 165 will become parallel to one another and the distance between the elbow of simulated robotic arm 165 and the elbow of the simulated robotic arm 166 decreases. Conversely as the user manipulates the left hand controller 201 and the right hand controller 202 to bend the robotic arms 42A and 42B, so that the distance between the virtual elbows 128 of the robotic arms 42A and 42B are is further apart, simulated robotic arms 166 and 165 will not be parallel to one another and the distance between the elbow of simulated robotic arm 165 and the elbow of the simulated robotic arm 166 will increase. The robot pose views 171 and 172 provide the user with the spatial awareness during a surgical procedure, because the live video footage 168 does not provide visualization of the entire length of the robotic arms 42A and 42B.

In FIG. 7, the simulated robotic arms 191 and 192 are shown as being within the camera assembly 14 field of view associated with frustum 151, which provides the user with a situational awareness and spatial awareness of where the robotic arm 42B and the robotic arm 42A are located or positioned within a portion of the actual cavity captured of the patient. The camera view associated with the robot pose view 171 is a simulated view of the robotic arm 42B and the robotic arm 42A as if the user were viewing the actual view of the robotic arm 42B and the robotic arm 42A from a side view within the cavity of the patient.

As noted above, the camera view can be the camera assembly 44 field of view which is the frustum 167. That is, the robot pose view 171 provides the user with a side view of simulated robotic arms 191 and 192, which are simulated views corresponding to the robotic arm 42B and the robotic arm 42A respectively.

In some embodiments, the graphical user interface 39 can display live video footage 168 from a single vantage point including a field of view of the cavity and the robotic arm 42B and the robotic arm 42A relative to different areas within the cavity as shown in FIG. 7. As a result, the user might not always be able to determine how the virtual elbows 128 of the robotic arm 42B and the robotic arm 42A are positioned. This is because the camera assembly 44 might not always include video footage of the virtual elbows 128 of the robotic arm 42B and video footage of the elbow of the robotic arm 42A, and therefore the user may not be able to determine how to adjust the right hand controller 202 and left hand controller 201 if they wish to maneuver within the cavity of the patient. The simulated view of the robotic arm 42B (robotic arm 191) and the simulated view of the robotic arm 42A (robotic arm 192) provides the user with a view point that allows the user to determine the positioning of the virtual elbows 128 of the robotic arm 42A and the robotic arm 42B because the left situational awareness camera view panel includes a simulated field of view of the entire length of the robotic arms 191 and 192. Because the simulated field of view of the robot pose view 171 includes a view of the virtual elbows 128 of robotic arms 191 and 192, the user can adjust the positioning of the robotic arm 42B and the robotic arm 42A by manipulating the left hand controller 201 and the right hand controller 202, and watching how the robotic arms 191 and 192 move in accordance with the manipulation of the left hand controller 201 and the right hand controller 202.

The graphical user interface 39 can include the robot pose view 172 within which there is a frustum 167 that is the field of view, of the camera assembly 44, associated with a portion of the cavity of the patient, and the robotic arms 165 and 166 with a simulated camera 158 and simulated robotic supporting arm supporting the robotic arms 165 and 166.

In FIG. 7 the simulated robotic arms 165 and 166 are shown as being within the frustum 167, which is representative of location and positioning of the robotic arm 42B and the robotic arm 42A within the actual cavity of the patient. The view shown in the robot pose view 172 is a simulated view of the robotic arm 42B and the robotic arm 42A as if the user were viewing the robotic arm 42B and the robotic arm 42A from a top down view within the cavity of the patient. That is, the robot pose view 172 provides the user with a top down view of the simulated robotic arms 165 and 166, which are simulated views corresponding to the robotic arm 42B and the robotic arm 42A, respectively. The top down view provides the user with the ability to maintain a certain level of situational awareness of the robotic arm 42B and the robotic arm 42A as the user is performing a procedure within the cavity. The view of the simulated robotic arm 165 corresponding to the robotic arm 42B and the view of simulated robotic arm 166 corresponding to the robotic arm 42A provides the user with a top view perspective that allows them to determine the positioning of the robotic arm 42B and the robotic arm 42A, because the robot pose view 172 includes a simulated top-down field of view of the robotic arms 165 and 166, the camera 158, as well as support arm of the robotic assembly. Because the simulated field of view of the camera assembly 44 as outlined by the frustum 167 includes a top-down view of the simulated robotic arms 165 and 166, the user can adjust the positioning of the robotic arm 42B and the robotic arm 42A by manipulating the left hand controller 201 and the right hand controller 202, and watching how the simulated robotic arms 165 and 166 move forward or move backward within a portion of the cavity within the frustum 167 in accordance with the manipulation of the left hand controller 201 and the right hand controller 202.

The simulated view of the robotic arms 42B and 42A in the robot pose views 171 and 172 is automatically updated to stay centered on the trocar 50 while maintaining the robotic arms 42B and 42A in view. In some embodiments, this can be accomplished based on one or more sensors, from the sensing and tracking module 16 that are on the robotic arms 42B and 42A, providing information to the right hand controller 202 and the left hand controller 201. The sensors can be an encoder or hall effect sensor or other suitable sensor.

### Inserting a Robotic Assembly

Articulated robotic arm insertion as taught herein can be employed with any of the surgical robotic systems taught above or any other suitable surgical robotic system. Further, some embodiments taught herein can be employed with semi-robotic endoscopic surgical systems that are only robotic in part.

Articulated robotic arm insertion (also referred to as a “primary methodology”) can be understood with reference to embodiments depicted in FIGS. 8-15 described below. For convenience, like reference numbers are used to reference similar features of the various embodiments shown in the figures, unless otherwise noted.

FIG. 8 is a flowchart illustrating steps 300 for inserting a robotic assembly carried out by the surgical robotic system 10 of the present disclosure. FIG. 8 is a flowchart based on a robotic arm having three articulated joints, wrist joint, elbow joint and shoulder joint. Nonetheless, the process associated with the flowchart of FIG. 8 is applicable to a robotic arm having more than three articulated joints.

In step 302, the surgical robotic system 10 enters an insertion mode allowing a user to insert the camera assembly 44 and the robotic arm assembly 42 of the robotic assembly 20 through the trocar 50 into an interior cavity of a subject. In some embodiments, a user can control the foot pedal array 19 to enter an insertion mode. In some embodiments, a user can control one of the hand controllers 201/202 to enter a menu mode and the surgical robotic system 10 can display a menu on the display 12. The user can control the appropriate hand controller 201/202 to select an insertion mode on the menu. In some embodiments, the surgical robotic system 10 determines that the instrument tips 120 are installed properly (e.g., via data obtained from sensors associated with the instrument tips 120 or via a user input) and, in turn, the surgical robotic system 10 then automatically enters an insertion mode. It should be understood that a user can control one or both of hand controllers or one or both of the foot petals to enter an insertion mode.

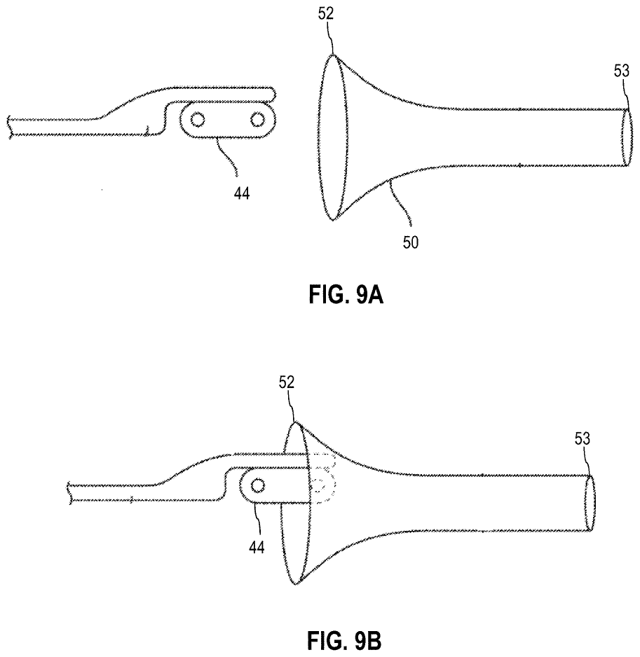

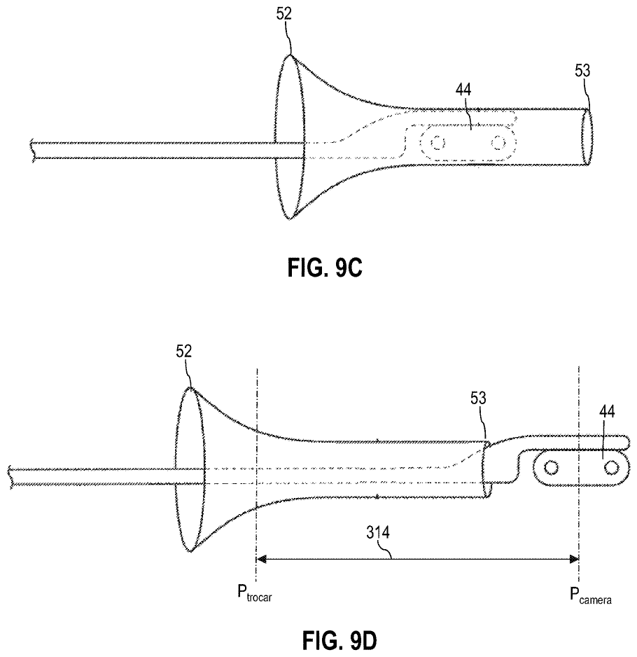

In step 304, the user via the surgical robotic system 10 inserts the camera assembly 44 through the trocar 50. Examples are described with respect to FIGS. 9A-9D.

In step 306, the user via the surgical robotic system 10 controls the camera assembly 44 to reach a desired camera view. For example, after the camera assembly 44 reaches a camera insertion point in the interior cavity as described with respect to FIGS. 9A-9D, the surgical robotic system 10 can automatically deploy the camera assembly 44 to face forward into an interior cavity, for example an abdominal space (e.g., a camera deployed state). If a current camera view is not sufficient, the surgical robotic system 10 allows the user to adjust the position and orientation of the camera assembly 44 via pitch and yaw controls of the camera support arm to reach a desired camera view.

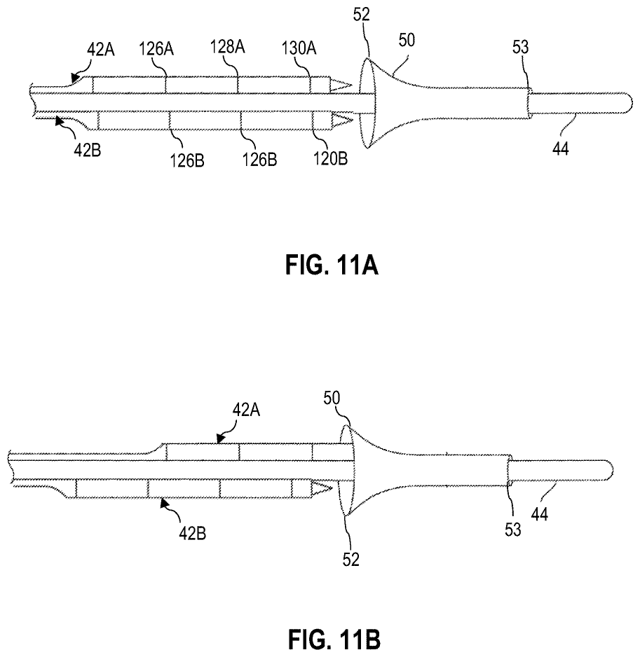

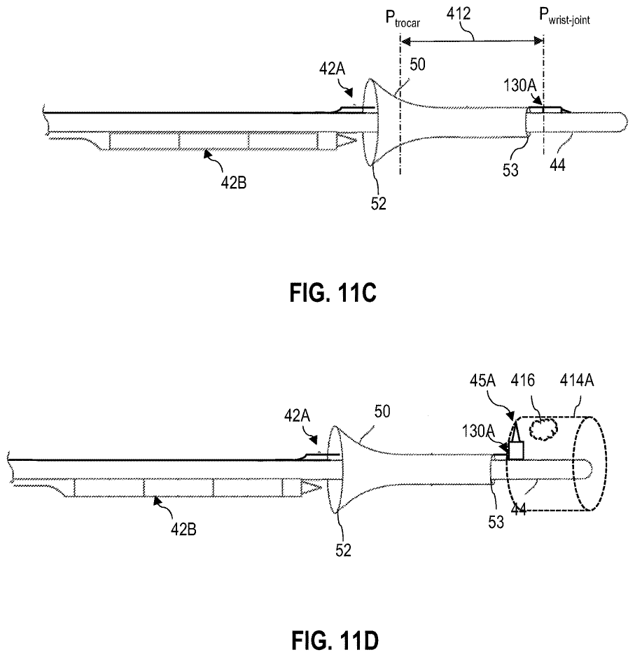

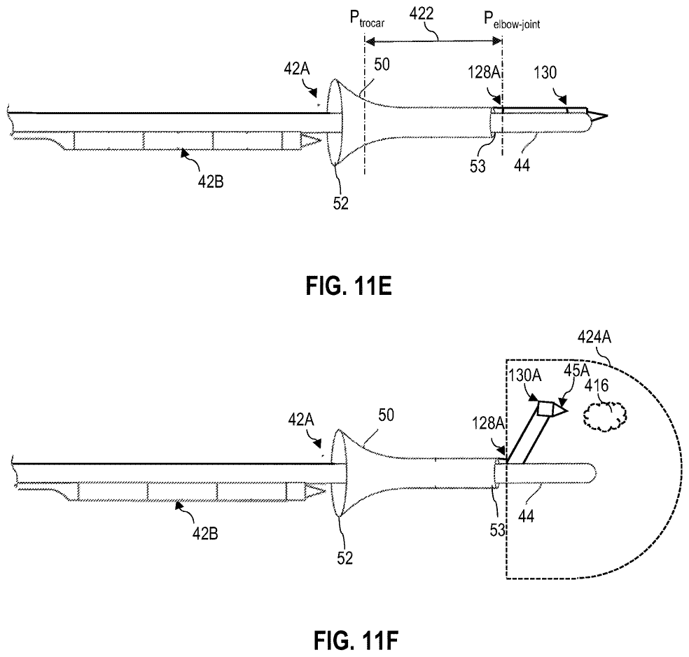

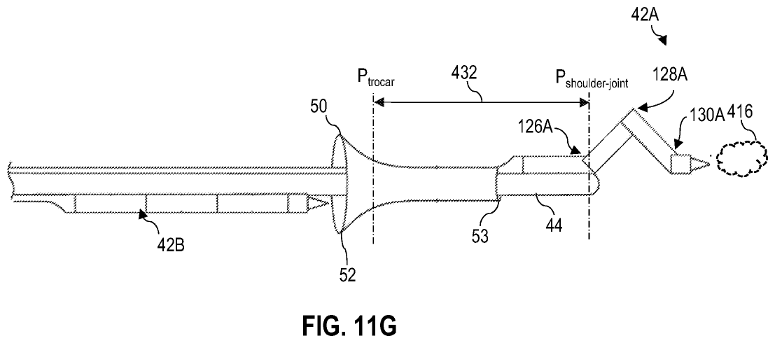

In step 308, the surgical robotic system 10 determines that a first articulated joint 130 of the plurality of articulated joints exits the trocar 50 and reaches a first articulated joint inserted position in the interior cavity. The first articulated joint inserted position indicates that the first articulated joint is past the second terminal end 53 of the trocar 50 and is free to rotate relative thereto. FIGS. 11A-11D and the text associated therewith provide more details on the articulated robotic arm insertion of the first articulated joint 130 as taught herein.

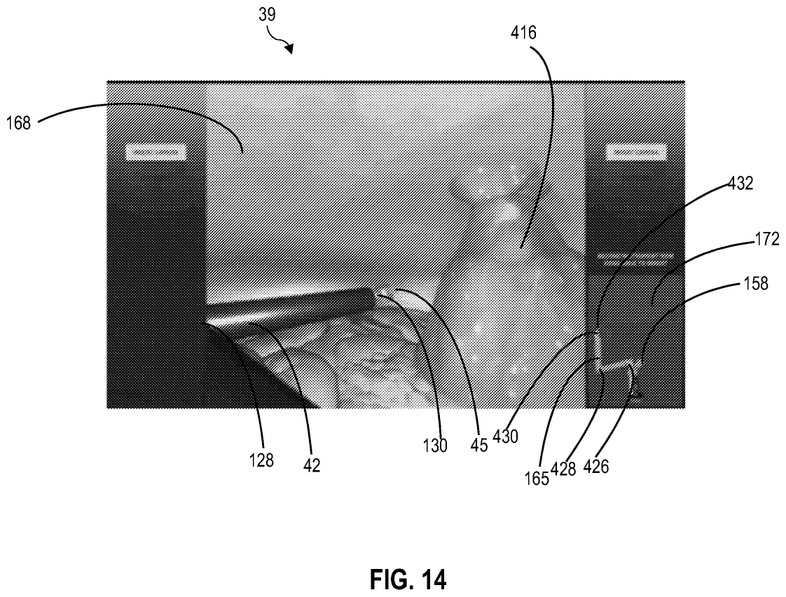

In step 310, the surgical robotic system 10 enables articulation of the first articulated joint 130 within a first volume 414 in the interior cavity such that the first articulated joint 130 is able to be articulated by one or both of the hand controllers 201/202 within the first volume 414. In some embodiments, the surgical robotic system 10 enables articulation of the first articulated joint 130 automatically, for example, after reaching the minimum insertion point. In some embodiments, the user of the surgical robotic system 10 enables articulation of the first articulated joint 130 via the hand controller(s) 201/202 or the foot petal(s) 19, or the graphical user interface 39 after reaching the minimum insertion point. FIGS. 11D and 14 and the text associated therewith provide more details on the articulation of the first articulated joint 130 as taught herein.

In step 312, the surgical robotic system 10 determines that a second articulated joint 128 of the plurality of articulated joints exits the trocar 50 and reaches a second articulated joint inserted position past the second terminal end 53 of the trocar 50 in the interior cavity. The second articulated joint inserted position indicates that the second articulated joint 128 is free to rotate relative to the second terminal end 53 of the trocar 50. FIGS. 11E and 11F and the text associated therewith provide more details on the articulated robotic arm insertion of the second articulated joint 128 as taught herein.

In step 314, the surgical robotic system 10 enables articulation of the second articulated joint 128 within a second volume 424 in the interior cavity such that the second articulated joint 128 is able to be articulated by one or both of the hand controllers 201/202 within the second volume 424. In some embodiments, the surgical robotic system 10 enables articulation of the second articulated joint 128 automatically, for example, after reaching the minimum insertion point. In some embodiments, the user of the surgical robotic system 10 enables articulation of the second articulated joint 128 via the hand controller(s) 201/202 or the foot petal(s) 19, or the graphical user interface 39 after reaching the minimum insertion point. FIGS. 11F and 14 and the text associated therewith provide more details on the articulation of the second articulated joint 128 as taught herein.

In step 316, the surgical robotic system 10 determines that a third articulated joint 126 of the plurality of articulated joints exits the trocar 50 and reaches a third articulated joint inserted position past the second terminal end 53 of the trocar 50 in the interior cavity. The third articulated joint inserted position indicates that the third articulated joint 126 is free to rotate relative to the trocar 50. FIGS. 11G and 14 and the text associated therewith provide more details on the articulated robotic arm insertion of the third articulated joint 126 as taught herein.

In step 318, the surgical robotic system 10 enables articulation of the third articulated joint 128 within the interior cavity such that the third articulated joint 126 is able to be articulated by one or both of the hand controllers 201/202 within the interior cavity. In some embodiments, the surgical robotic system 10 enables articulation of the third articulated joint 126 automatically, for example, after reaching the minimum insertion point. In some embodiments, the user of the surgical robotic system 10 enables articulation of the third articulated joint 126 via the hand controller(s) 201/202 or the foot petal(s) 19, or the graphical user interface 39 after reaching the minimum insertion point.

In step 320, the surgical robotic system 10 determines that the first robotic arm 42A is fully inserted into the interior cavity. FIGS. 11G and 14 and the text associated therewith provide more details on the articulated robotic arm insertion as taught herein.

In step 322, the surgical robotic system 10 enables, automatically or via input from the user one of the hand controllers 201/202, or the foot petal(s) 19, or the graphical user interface 39 full articulation of the first robotic arm 42A. Examples are described with respect to FIGS. 11G and 14 and the text associated therewith provide more details on the articulated robotic arm insertion as taught herein.

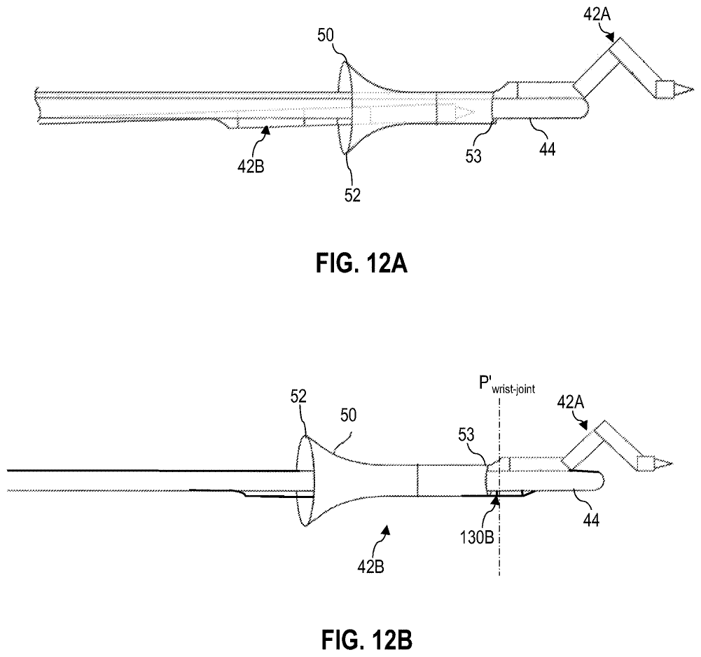

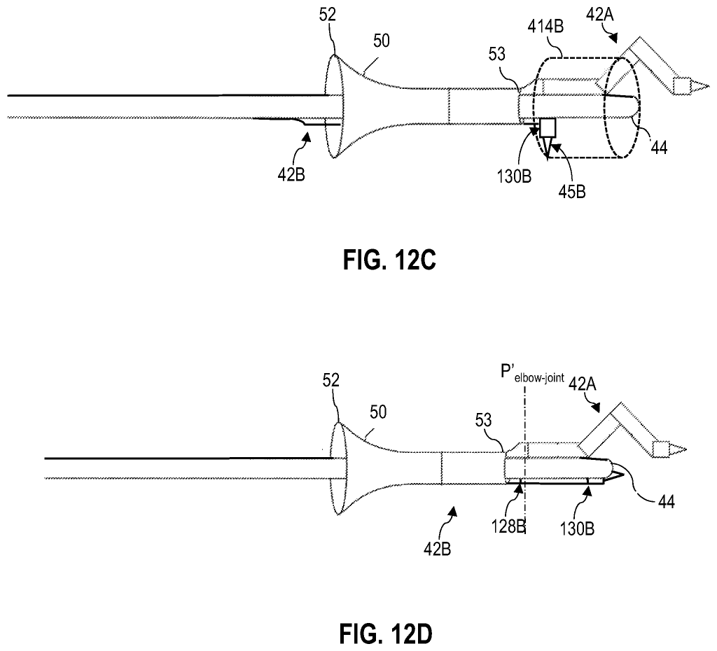

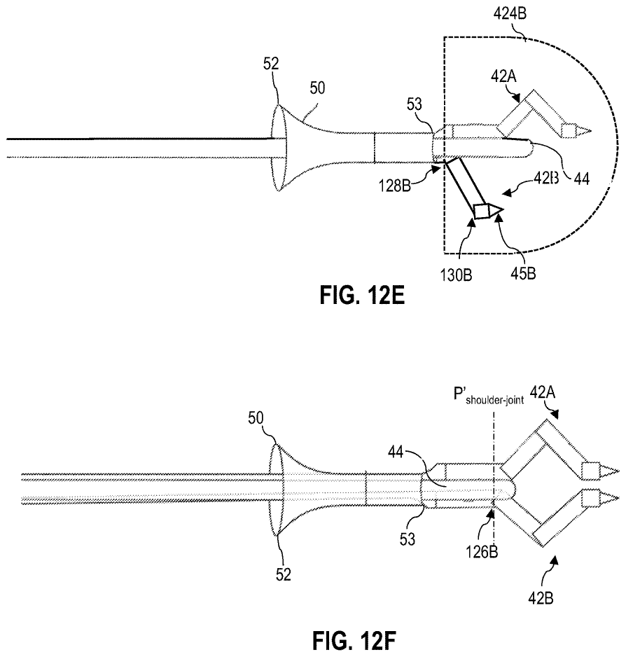

In step 324, if the surgical robotic system 10 includes a second robotic arm 42B and if the user desires the use of the second robotic arm 42B, the user via the surgical robotic system 10 inserts the second robotic arm 42B of the robotic arm assembly 42 through the trocar 50 into the interior cavity, otherwise the surgical robotic system 10 can proceed to step 326. The articulated robotic arm insertion of the second robotic arm 42B can repeat the steps 308-322. Examples are described with respect to FIGS. 12-14 and the text associated therewith provide more details on the articulated robotic arm insertion of the second robotic arm 42B as taught herein.

In step 326, the surgical robotic system 10 determines that the insertion process is complete. For example, if the surgical robotic system 10 can determine that both the camera assembly and the robotic arm assembly are fully inserted in desired locations (e.g., articulated joint inserted positions, target locations or the like) within the cavity, the surgical robotic system 10 can determine that the insertion process is complete. The surgical robotic system 10 can also allow the user to exit the insertion mode using the onscreen menu, the foot pedals and/or the hand controllers. Those skilled in the art will appreciate that some or all of the above steps may be repeated depending on the number of articulated joints included in a robotic arm.

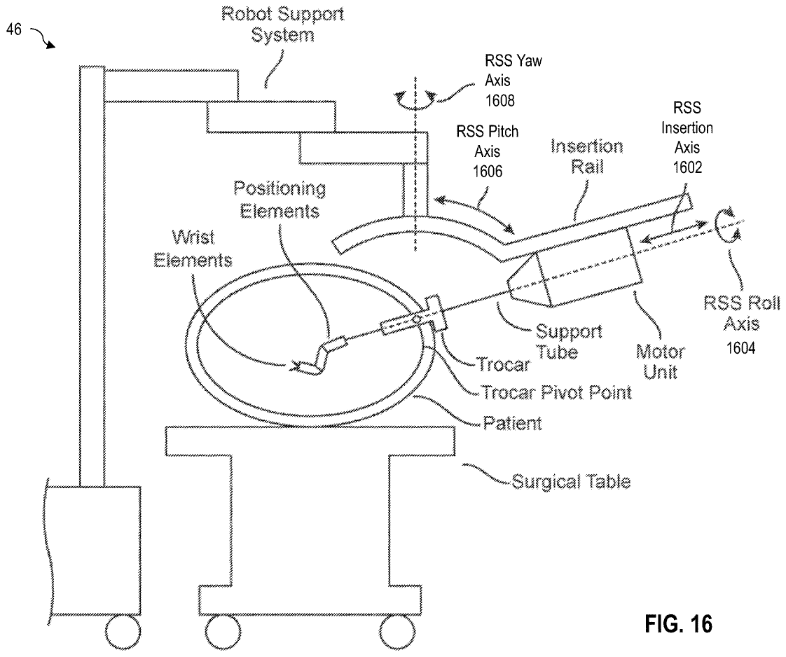

If at any time during the process of performing steps 302-324 it is determined that articulation of the first and/or second robotic arm is not sufficient to avoid an obstruction and/or sensitive tissue, the operator can take further steps as described below in relation to FIGS. 16-18 to reposition the first and/or the second volume in the internal cavity.

FIGS. 9A-9D illustrate insertion of the camera assembly 44 through the trocar 50 in accordance with some embodiments. As shown in FIGS. 9A-9C, the camera assembly 44 is being inserted into the trocar 50 (e.g., using one or both of the hand controllers 201/202 and/or the foot pedals 19) at a first terminal end 52 of the trocar 50. For example, a user can control one or both of the hand controllers 201/202 to select an insertion mode on a displayed menu and use the hand controllers 201/202 to control a position and an orientation of the camera assembly 44 in the interior cavity. In some embodiments, the user can use foot pedals 19 to select an insertion mode such that the hand controllers 201/202 can control the position and the orientation of the camera assembly 44 in the trocar 50 or in an interior cavity. As shown in FIG. 9D, the camera assembly 44 exits at a second terminal end 53 of the trocar 50 and reaches a camera inserted position P_{camera} in the interior cavity. The camera inserted position P_{camera} can be a minimally inserted position that is a position allowing a distance 314 between a trocar position P_{trocar} (e.g., a center or a proximal portion of the trocar 50) and a position of the camera assembly 44 satisfying a distance threshold (e.g., equal to or greater than the distance threshold). A distance threshold indicates that the camera assembly 44 has reached a minimum distance past the second terminal end 53 of the trocar 50 to allow the camera assembly 44 to freely rotate relative to the trocar 50. When the camera assembly 44 has successfully moved to the minimally inserted position, the camera assembly 44 can be automatically deployed to face forward into the interior cavity (e.g., camera deployed state), for example, an abdominal space. If the camera view is not sufficient after automatically deploying to face forward, a user can control the foot pedal 19 or other controls to enter the camera view mode 251 such that the user is able to use the hand controllers 201/202 to control the position and orientation of the camera assembly 44 as well as a pitch and a yaw of the camera assembly 44 to reach a desired camera view. Once the camera is in a deployed state, a scan mode may be automatically or manually initiated to capture an overview of the interior cavity. This allows the user to determine whether any features within the cavity are blocking access to a surgical site and determine whether such features require avoiding or cutting prior to fully inserting one or both of the robotic arms.

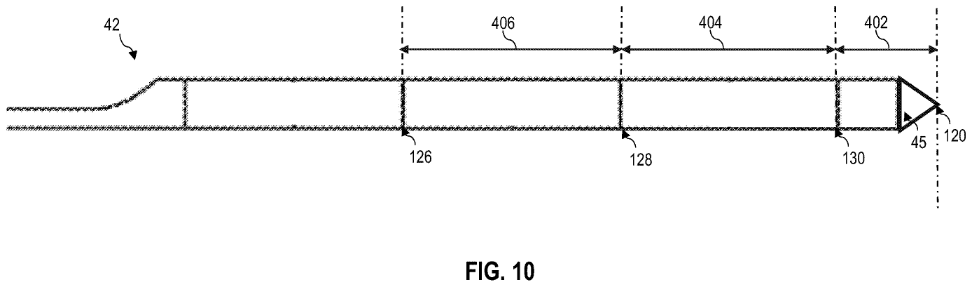

To facilitate explanation of the articulated robotic arm insertion process as taught herein, FIG. 4B is redrawn in a simplified manner to help explain a hand length, a forearm length and an upper arm length of a robotic arm of the robotic arm assembly 42. As shown in FIG. 10 articulated joints along the robotic arm of the robotic arm assembly 42 are spaced as follows. The robotic arm assembly 42 includes the shoulder joint 126, the elbow joint 128, the wrist joint 130, and the end-effector 45 having the instrument tip 120. The shoulder joint 126, the elbow joint 128, the wrist joint 130 can include a series of hinge and rotary joints to provide each robotic arm 42 with positionable, seven degrees of freedom, along with one additional grasping degree of freedom for the end-effector 45 in some embodiments. A hand length 402 extends from the instrument tip 120 to the wrist joint 130. A forearm length 404 extends from the wrist joint 130 to the elbow joint 128. An upper arm length 406 extends from the elbow joint 128 to the shoulder joint 126.

FIGS. 11A-11G illustrate an articulated robotic arm insertion of the first robotic arm 42A through the trocar 50 in accordance with some embodiments. As shown in FIGS. 11A and 11B, after the camera assembly 44 is inserted into the trocar 50 and reaches the camera inserted position P_{camera} (shown in FIG. 9D), if the view of the camera assembly 44 indicates that the articulated robotic arm insertion process can continue, the first robotic arm 42A is inserted into the trocar 50 (e.g., using hand controllers 201/202 and/or foot pedals 19). In some embodiments, a user can be alerted when it is possible to begin insertion of a robotic arm 42. For example, the display 12 or the graphical user interface 39 can output an alert (e.g., visual or audio output) indicating that a user is able to begin insertion of a robotic arm 42. As another example, the surgical robotic system 10 can alert a user using sound and/or haptic effect. The user can select one of robotic arms (e.g., the first robotic arm 42A) and begin to insert the first robotic arm 42A using the hand controllers 201/202 to control a position and an orientation of the first robotic arm 42A into the interior cavity through the trocar 50. In some embodiments (not shown), a user can insert the first robotic arm 42A before the camera assembly 44 is inserted into the interior cavity. For example, a user can control one or both of the hand controllers 201/202 to select an insertion mode on a displayed menu and control one or both of the hand controllers 201/ 202 to control a position and an orientation of the first robotic arm 42A into an interior cavity through the trocar 50. In some embodiments, a user can control the foot pedal 19 to enter the insertion mode.

As shown in FIG. 11C, the wrist joint 130A exits the trocar 50 and reaches a wrist joint inserted position P_{wrist-joint} in the interior cavity. The wrist joint inserted position P_{wrist-joint} indicates that the wrist joint 130A is sufficiently past the second terminal end 53 of the trocar 50 and free to rotate relative to the second terminal end 53 of the trocar 50. The wrist joint inserted position P_{wrist-joint} refers to a position allowing a distance 412 between the trocar position P_{trocar} and a position of the wrist joint 130A satisfying a wrist articulation distance threshold (e.g., equal to or greater than the wrist articulation distance threshold). A wrist articulation distance threshold indicates that the wrist joint 130A has reached a minimum distance past the second terminal end 53 of the trocar 50 to allow the wrist joint 130A to freely rotate relative to the trocar 50.

As shown in FIG. 11D, once the wrist joint 130A reaches the wrist joint inserted position P_{wrist-joint} articulation of the wrist joint 130A is enabled either automatically or via an action taken by the user with, for example, one of the hand controllers (e.g., the hand controller 201 or 202 shown in FIG. 6B). Articulation of the wrist joint 130A at this point in the articulated robotic arm insertion process is limited to a first volume 414A in the interior cavity. The wrist joint 130A is able to be articulated by one or both of the hand controllers 202/202 within the first volume 414A (e.g., via rotation translation, pitch, yaw, roll or other suitable movement that changes a position and/or an orientation of the wrist joint 130A). The first volume 414A defines a volume accessible by wrist articulation once inserted to or past the wrist joint inserted position P_{wrist-joint}. The first volume 414A is determined by the hand length 402 and the forearm length 404 (as shown in FIG. 10). For example, the first volume 414A can be a cylinder with a radius of the hand length 402 and a height of the forearm length 404, e.g., Pi*hand length*hand length*forearm length. A user can use the wrist joint 130A and the end-effector 45A to manipulate tissue 416 within the first volume 414A to clear the way for further arm insertion and/or avoid an obstruction (e.g., abdominal obstruction). In similar fashion, a user can articulate the wrist joint 130A to steer the end-effector 45A past an obstruction (e.g., abdominal obstruction) in the first volume 414A. In some embodiments, as the wrist joint 130A and the end-effector 45A become articulated, the wrist joint 130A may not translate other than further insertion, but the end-effector 45A may be moved to match the orientation of the hand controllers 201/202, opened and closed controlled by the user.

As shown in FIG. 11E, the user can decide to continue insertion of the robotic arm 42A, in which case the elbow joint 128A exits the second terminal end 53 of the trocar 50 and reaches an elbow joint inserted position P_{elbow-joint} in the interior cavity. The elbow joint inserted position P_{elbow-joint} indicates that the elbow joint 128A is sufficiently past the second terminal end 53 of the trocar 50 and free to rotate relative to the trocar 50. The elbow joint inserted position P_{elbow-joint} can refer to a position allowing a distance 422 between the trocar position P_{trocar} and a position of the elbow joint 128A satisfying an elbow articulation distance threshold (e.g., equal to or greater than the elbow articulation distance threshold). The elbow articulation distance threshold indicates that the elbow joint 128A has reached a minimum distance past the second terminal end 53 of the trocar 50 to allow the elbow joint 128A to freely to rotate relative to the trocar 50. Articulation of the elbow joint 128A once it reaches the minimum distance may be enabled automatically by the robotic surgical system 10 or enabled by a user of the robotic surgical system 10.

As shown in FIG. 11F, articulation of the elbow joint 128A takes place within a second volume 424A in the interior cavity such that the elbow joint 128A is able to be articulated by one or both of the hand controllers 201/202 within the second volume 424A (e.g., via rotation translation, pitch, yaw, roll or other suitable movement that changes a position and/or an orientation of the elbow joint 128A). The second volume 424A defines a volume accessible by wrist and elbow articulations once inserted to or past the elbow joint inserted position P_{elbow-joint.} The second volume 424A is defined by the hand length 402, the forearm length 404, and the upper arm length 406 (as shown in FIG. 10). For example, the second volume 424A can be a combination of a cylinder with a radius of a sum (as referred to as “elbow reach”) of the hand length 402 and forearm length 404 and a height of the upper arm length 402 and a hemisphere beyond that cylinder. The hemisphere can have the elbow reach as its radius. The second volume 424A can be calculated by (Pi*elbow reach*elbow reach*upper arm length)+(Pi*⅔*elbow reach*elbow reach*elbow reach). A user can use one or both of the hand controllers 201/202 to control the elbow joint 128A, the wrist joint 130A and the end-effector 45A to manipulate tissue 416 within the second volume 424A to clear the way for further arm insertion and/or avoid an obstruction 416 (e.g., abdominal obstruction). In similar fashion, a user can articulate the elbow joint 128A and/or the wrist joint 130A to steer the end-effector 45A past an obstruction (e.g., abdominal obstructions) in the second volume 424A. In some embodiments, the wrist joint 130A may translate further along the insertion and sideways by as much as the forearm length 404. The articulations of the elbow joint 128A and the wrist joint 130A can be used to position the end-effector 45A (e.g., the location and orientation of the end-effector 45A) in accordance with the movements of one or both of the hand controllers 201/202 held by the user, e.g., including opening and closing the end-effector 45A. In some embodiments, insertion of the robotic arm 42A past the wrist joint 130A but not as far as the shoulder joint 126A may be sufficient for the user to perform the desired task.

As shown in FIG. 11G, the user can decide to continue with insertion of the robotic arm 42A, in which case the shoulder joint 126A exits the second terminal end 53 of the trocar 50 and reaches a shoulder joint inserted position P_{shoulder-joint} in the interior cavity. The shoulder joint inserted position P_{shoulder-joint} can refer to a position allowing a distance 432 between the trocar position P_{trocar} and a position of the shoulder joint 126A satisfying a shoulder articulation distance threshold (e.g., equal to or greater than the shoulder articulation distance threshold). The shoulder articulation distance threshold indicates that the shoulder joint 126A has reached a minimum distance past the second terminal end 53 of the trocar 50 to allow the shoulder joint 126A to freely rotate relative to the trocar 50.

When the shoulder joint 126A is free to rotate relative to the trocar 50, the surgical robotic system 10 can determine that the first robotic arm 42A is fully inserted into the interior cavity and determine that the first robotic arm 42A can be fully articulated. For example, full articulation of the first robotic arm 42A including the shoulder joint 126A, the elbow joint 128A, the wrist joint 130A, the end-effector 45A, and/or other components of the first robotic arm 42A is enabled via one or both of the hand controllers 201/202 within the interior cavity such that a user can use one or both of the hand controllers 201/202 to control the first robotic arm 42A to manipulate the tissue 416 or avoid the obstruction 416.

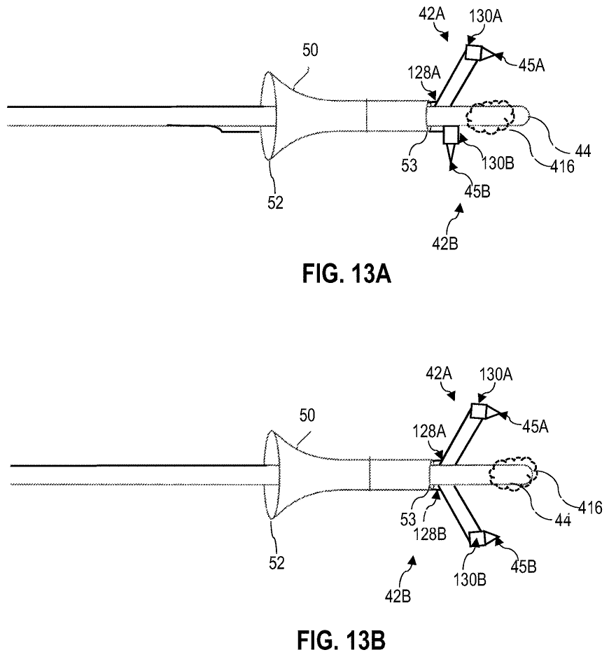

FIGS. 12A-12G illustrate an articulated robotic arm insertion of the second robotic arm 42B through the trocar 50 in accordance with some embodiments. The articulated robotic arm insertion process of the second robotic arm 42B is similar to the articulated robotic arm insertion process of the first robotic arm 42A as shown in FIGS. 11A-11G. After the first robotic arm 42A is inserted into the interior cavity, a user can begin an articulated robotic arm insertion of the second robotic arm 42B. In some embodiments, the robotic arms 42 can be fully or partially inserted in any order or in a specific order as described with respect to FIGS. 13A and 13B.

As shown in FIGS. 12A-12C, the wrist joint 130B of the second robotic arm 42B exits the trocar 50 and reaches a wrist joint inserted position P′_{wrist-joint} of the second robotic arm 42B in the interior cavity. The wrist joint inserted position P′_{wrist-joint} indicates that the wrist joint 130B is sufficiently past the second terminal end 53 of the trocar 50 and free to rotate relative to the second terminal end 53 of the trocar 50. The wrist joint inserted position P′_{wrist-joint} can be determined based on a wrist articulation distance threshold as described above with respect to FIG. 12C. While the first robotic arm 42A is fully articulated, articulation of the wrist joint 130B can be enabled via one of both of the hand controllers 201/202 within a first volume 414B associated with the second robotic arm 42B. The first volume 414B can be calculated as described above with respect to FIG. 11D.

As shown in FIGS. 12D and 12E, the user can decide to continue insertion of the robotic arm 42B in which case the elbow joint 128B of the second arm 42B exits the second terminal end 53 of the trocar 50 and reaches an elbow joint inserted position P′_{elbow-joint} of the second robotic arm 42B in the interior cavity. The elbow joint inserted position P′_{elbow-joint} indicates that the elbow joint 128B is sufficiently past the second terminal end 53 of the trocar 50 and free to rotate relative to the trocar 50. The elbow joint inserted position P′_{elbow-joint} can be determined based on an elbow articulation distance threshold as described above with respect to FIG. 11F. Articulation of the elbow joint 128B can be enabled via one or both of the hand controllers 201/202 within a second volume 424B associated with the second robotic arm 42B. The second volume 424B can be calculated as described above with respect to FIG. 11F.

As shown in FIG. 12F, the user can decide to continue insertion of the robotic arm 42B, in which case the shoulder joint 126B exits the second terminal end 53 of the trocar 50 and reaches a shoulder joint inserted position P′_{shoulder-joint} of the second robotic arm 42B in the interior cavity. The shoulder joint inserted position P′_{shoulder-joint} indicates that the shoulder joint 126B is sufficiently past the second terminal end 53 of the trocar 50 and free to rotate relative to the second terminal end 53 of the trocar 50. The shoulder joint inserted position P′_{shoulder-joint} can be determined based on a shoulder articulation distance threshold as described above with respect to FIG. 11G. When the shoulder joint 126B is free to rotate relative to the trocar 50, the second robotic arm 42B is fully inserted in the interior cavity.

In some embodiments, the surgical robotic system 10 can include more than two robotic arms. Each of the remaining robotic arms can be inserted into the interior cavity using the similar insertion process as the robotic arms 42. The surgical robotic system 10 can determine that the insertion process is complete if all of the robotic arms 42 and/or the camera assembly 44 are fully inserted in desired locations within the interior cavity. The surgical robotic system 10 can operate the display 12 to output one or more selectable menu items allowing the user to exit the insertion mode.

In some embodiments, the camera assembly 44 and the robotic arm assembly 42, or robotic arms of the robotic arm assembly 42 can be fully or partially inserted in any order or in a specific order. For example, the second robotic arm 42B can be followed by the camera assembly 44 and then followed by the first robotic arm 42A. A robotic arm 42 can be inserted during the insertion process of the camera assembly 44 (e.g., the camera assembly 44 is partially inserted) or during insertion process of another robotic arm 42 (e.g., the robotic arm 42 is partially inserted). Examples are shown in FIGS. 13A and 13B.

FIGS. 13A and 13B illustrate an articulated robotic arm insertion of a robotic arm while another robotic arm is partially inserted. The first robotic arm 42A or the second robotic 42B can be partially inserted into the interior cavity while the other robotic arm is also partially inserted. As shown in FIG. 13A, a user can decide to insert and articulate which robotic arms 42 and which joints of the robotic arms 42, in which case the elbow joint 128A, the wrist joint 130A, and the wrist joint 130B are inserted into the interior cavity and exit the second terminal end 53 of the trocar 50 and reach the elbow joint inserted position P_{elbow-joint} (e.g., shown in FIG. 11E), the wrist joint inserted position P_{wrist-joint} (e.g., shown in FIG. 11C), and the wrist joint inserted position P′_{wrist-joint} (e.g., shown in FIG. 12B). Articulation of the elbow joint 128A, the wrist joint 130A, and the wrist joint 130B take place in the interior cavity such that these joints are able to be articulated by one or both of the hand controllers 201/202 (e.g., via rotation translation, pitch, yaw, roll or other suitable movement that changes a position and/or an orientation of the elbow joint 128A, the wrist joint 130A, the end-effector 45A, the wrist joint 130B, and the end-effector 45B). The user can use one or both of the hand controllers 201/202 to control the elbow joint 128A, the wrist joint 130A, the end-effector 45A, the wrist joint 130B, and/or the end-effector 45B to manipulate tissue 416 to clear the way for further arm insertion and/or avoid an obstruction 416 (e.g., abdominal obstruction). In similar fashion, the user can articulate the elbow joint 128A, the wrist joint 130A, and/or the wrist joint 130B, to steer one or both of the end-effectors 45 past an obstruction (e.g., abdominal obstructions).

As shown in FIG. 13B, the user can decide to insert and articulate which robotic arms 42 and which joints of the robotic arms 42, in which case the elbow joint 128A, the wrist joint 130A, the elbow joint 128B, and the wrist joint 130B are inserted into the interior cavity and exit the second terminal end 53 of the trocar 50 and reach the elbow joint inserted position P_{elbow-joint} (e.g., shown in FIG. 11E), the wrist joint inserted position P_{wrist-joint} (e.g., shown in FIG. 11C), the elbow joint inserted position P′_{elbow-joint} (e.g., shown in FIG. 12D), and the wrist joint inserted position P′_{wrist-joint} (e.g., shown in FIG. 12B). Articulation of the elbow joint 128A, the wrist joint 130A, the elbow joint 128B, and the wrist joint 130B take place in the interior cavity such that these joints are able to be articulated by one or both of the hand controllers 201/202 (e.g., via rotation translation, pitch, yaw, roll or other suitable movement that changes a position and/or an orientation of the elbow joint 128A, the wrist joint 130A, the end-effector 45A, the elbow joint 128B, the wrist joint 130B, and the end-effector 45B). The user can use one or both of the hand controllers 201/202 to control the elbow joint 128A, the wrist joint 130A, the end-effector 45A, the elbow joint 127B, the wrist joint 130B, and/or the end-effector 45B to manipulate tissue 416 at a different location to clear the way for further arm insertion and/or avoid an obstruction 416 (e.g., abdominal obstruction). In similar fashion, the user can articulate the elbow joint 128A, the wrist joint 130A, the elbow joint 128B, and/or the wrist joint 130B, to steer one or both of the end-effectors 45 past an obstruction (e.g., abdominal obstructions).

In some embodiments, at any time in the articulated robotic arm insertion process for each robotic arm 42 as described herein, the camera assembly 44 may be moved and reoriented so that a user can view the arm insertion and adjust the insertion path as needed. For example, during the insertion of the first robotic arm 42A as described with respect to FIGS. 11A-11G or during the insertion of the second robotic arm 42B as described with respect to FIGS. 12A-12F, a user can control the foot pedal 19 or one or both of the hand controllers 201/202 to enter a camera control mode to change a position and/or an orientation of the camera assembly 44 to obtain a desired camera view.

In some embodiments, as the robotic arm 42 advances through the trocar 50, a visual representation of the robotic arm 42 (“pose view”) can indicate that a robotic arm joint is able to be articulated by a hand controller 201/202 to change a position and an orientation. In some embodiments, the visual representation is color coded to represent which of the robotic arm joints is free to rotate within the interior cavity. A user may use the hand controller 201/202 to move the robotic arm joint indicated by the visual representation if necessary to avoid any abdominal obstructions. Examples are described below with respect to FIG. 14.

FIG. 14 depicts a graphical user interface 39 when a robotic arm is inserted into an interior cavity as taught herein. The graphical user interface 39 displays an image 168 (e.g., a live video footage) captured by the camera assembly 44 along a camera view. The image 168 includes the robotic arm 42 having the end-effector 45, the wrist joint 130, the elbow joint 128 (partially captured by the image 168). The graphical user interface 39 also includes the robot pose view 172 having a simulated robotic arm 165 corresponding to the robotic arm 42 and a simulated camera 158 corresponding to the camera assembly 44. The simulated robotic arm 165 includes a simulated shoulder joint 426 corresponding to the shoulder joint 126, a simulated elbow joint 428 corresponding to the elbow joint 128, a simulated wrist joint 430 corresponding to the wrist joint 130, a simulated end-effector 432 corresponding to the end-effector 45. The robot post view 172 is color coded in green indicating that the robotic arm 42 and the camera assembly 44 can be free to be manipulated. The robotic arm 42 can be controlled to manipulate the tissue 416 to clear the way for further arm insertion and/or avoid the obstruction 416 (e.g., incarcerated hernia or the like).

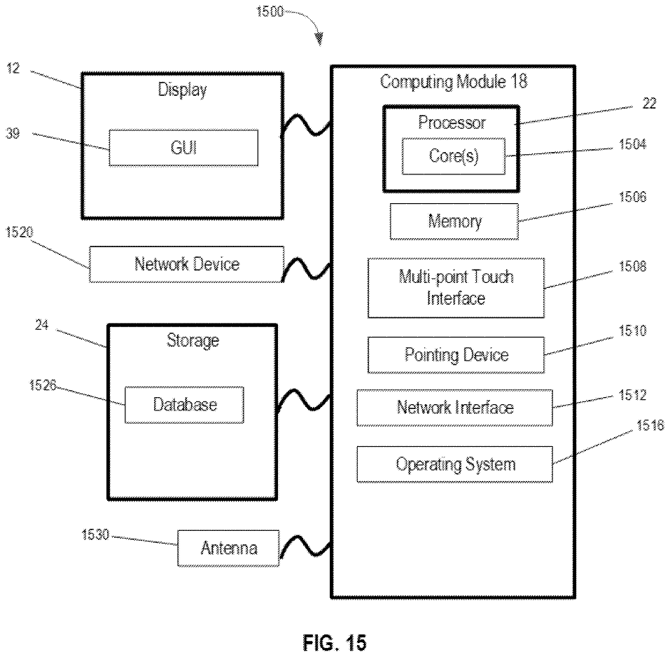

FIG. 15 schematically depicts the computing module 18 in more detail. Computing module 18 can be used to perform one or more steps of the methods provided by example embodiments. The computing module 18 includes one or more non-transitory computer-readable media for storing one or more computer-executable instructions or software for implementing example embodiments. The non-transitory computer-readable media can include, but are not limited to, one or more types of hardware memory, non-transitory tangible media (for example, one or more magnetic storage disks, one or more optical disks, one or more USB flashdrives), and the like. For example, memory 1506 included in the computing module 18 can store computer-readable and computer-executable instructions or software for implementing example embodiments. The computing module 18 also includes the processor 22 and associated core 1504, for executing computer-readable and computer-executable instructions or software stored in the memory 1506 and other programs for controlling system hardware. The processor 22 can be a single core processor or multiple core (1504) processor.

Memory 1506 can include a computer system memory or random access memory, such as DRAM, SRAM, EDO RAM, and the like. The memory 1506 can include other types of memory as well, or combinations thereof. A user can interact with the computing module 18 through the display 12, such as a touch screen display or computer monitor, which can display the graphical user interface (GUI) 39. The display 12 can also display other aspects, transducers and/or information or data associated with example embodiments. The computing module 18 can include other I/O devices for receiving input from a user, for example, a keyboard or any suitable multi-point touch interface 1508, a pointing device 1510 (e.g., a pen, stylus, mouse, or trackpad). The keyboard 1508 and the pointing device 1510 can be coupled to the visual display device 12. The computing module 18 can include other suitable conventional I/O peripherals.

The computing module 18 can also include one or more storage devices 24, such as a hard-drive, CD-ROM, or other computer readable media, for storing data and computer-readable instructions, applications, and/or software that implements example operations/steps of the surgical robotic system 10 as taught herein, or portions thereof, which can be executed to generate the graphical user interface 39 on the display 12. Example storage devices 24 can also store one or more databases for storing any suitable information required to implement example embodiments. The databases can be updated by a user or automatically at any suitable time to add, delete or update one or more items in the databases. Example storage device 24 can store one or more databases 1526 for storing provisioned data, and other data/information used to implement example embodiments of the systems and methods taught herein.

The computing module 18 can include a network interface 1512 configured to interface via one or more network devices 1520 with one or more networks, for example, Local Area Network (LAN), Wide Area Network (WAN) or the Internet through a variety of connections including, but not limited to, standard telephone lines, LAN or WAN links (for example, 802.11, T1, T3, 56 kb, X.25), broadband connections (for example, ISDN, Frame Relay, ATM), wireless connections, controller area network (CAN), or some combination of any or all of the above. The network interface 1512 can include a built-in network adapter, network interface card, PCMCIA network card, card bus network adapter, wireless network adapter, USB network adapter, modem or any other device suitable for interfacing the computing module 18 to any type of network capable of communication and performing the operations taught herein. Moreover, the computing module 18 can be any computer system, such as a workstation, desktop computer, server, laptop, handheld computer, tablet computer (e.g., the iPad® tablet computer), mobile computing or communication device (e.g., the iPhone® communication device), or other form of computing or telecommunications device that is capable of communication and that has sufficient processor power and memory capacity to perform the operations taught herein.

The computing module 18 can run any operating system 1516, such as any of the versions of the Microsoft® Windows® operating systems, the different releases of the Unix and Linux operating systems, any version of the MacOS® for Macintosh computers, any embedded operating system, any real-time operating system, any open source operating system, any proprietary operating system, any operating systems for mobile computing devices, or any other operating system capable of running on the computing device and performing the operations taught herein. In some embodiments, the operating system 1516 can be run in native mode or emulated mode. In some embodiments, the operating system 1516 can be run on one or more cloud machine instances.

The computing module 18 can also include an antenna 1530, where the antenna 1530 can transmit wireless transmissions a radio frequency (RF) front end and receive wireless transmissions from the RF front end.

A secondary methodology that relates to changing the pitch and/or yaw of the RSS 46 to relocate the actuation volume is now described. It should be appreciated that changes to the positioning of the RSS 46 during the secondary methodology can affect the foregoing articulated robotic arm insertion (i.e., the primary methodology). FIG. 16 shows one example of the RSS 46 comprising axes and translating positioning elements about a cavity, for example, an abdomen of a subject (e.g., patient) supported by a surgical table. The RSS 46 may facilitate the positioning and insertion of a robotic arm as described herein into the body cavity (e.g., abdomen) of a subject (e.g., a patient) at a trocar pivot point during a laparoscopic surgery. The RSS 46 may comprise a motor unit (e.g., motor 40) configured to control movement of a support tube (e.g., along an RSS insertion axis 1602 and/or RSS roll axis 1604) in order to insert the robotic arm into the cavity of the patient during laparoscopic surgery. The insertion may be guided by an insertion rail, whose movement may be controlled (e.g., along a RSS roll axis 1604, along a RSS pitch axis 1606 and/or a RSS yaw axis 1608) by the RSS 46. According to one or more embodiments described herein, the movement controlled by the RSS 46 may be on any combination of the RSS roll axis 1604, the pitch axis 1606, and/or the RSS yaw axis 1608. For example, in some cases the RSS 46 performs control along the pitch axis 1606 and/or the RSS yaw axis 1608 without providing control along the RSS roll axis 1604. Other combinations of roll/pitch/yaw control are possible in other embodiments.

The roll degree of freedom may be a rotation of the positioning elements via the support tube 122 about the lengthwise axis of the trocar (or another parallel axis). This may enable the orientation of the positioning elements to be adjusted to the operator's comfort of desire.

The yaw degree of freedom may be a rotation of the positioning elements via the support tube 122 about an axis perpendicular to the lengthwise axis of the trocar and typically perpendicular to the ground. This may enable the positioning elements to traverse left and right (relative to the trocar) and to slightly adjust their orientation.

The pitch degree of freedom may be a rotation of the positioning elements via the support tube 122 about an axis perpendicular to the lengthwise axis of the trocar and typically parallel to the ground. This may enable the positioning elements to traverse up and down (relative to the trocar) and to slightly adjust their orientation. As in both manual and robotic laparoscopy, both the yaw and pitch degrees of freedom may involve a rotation of the trocar relative to the patient, resulting in some temporary stretching of the patient's abdominal wall and surrounding tissue.

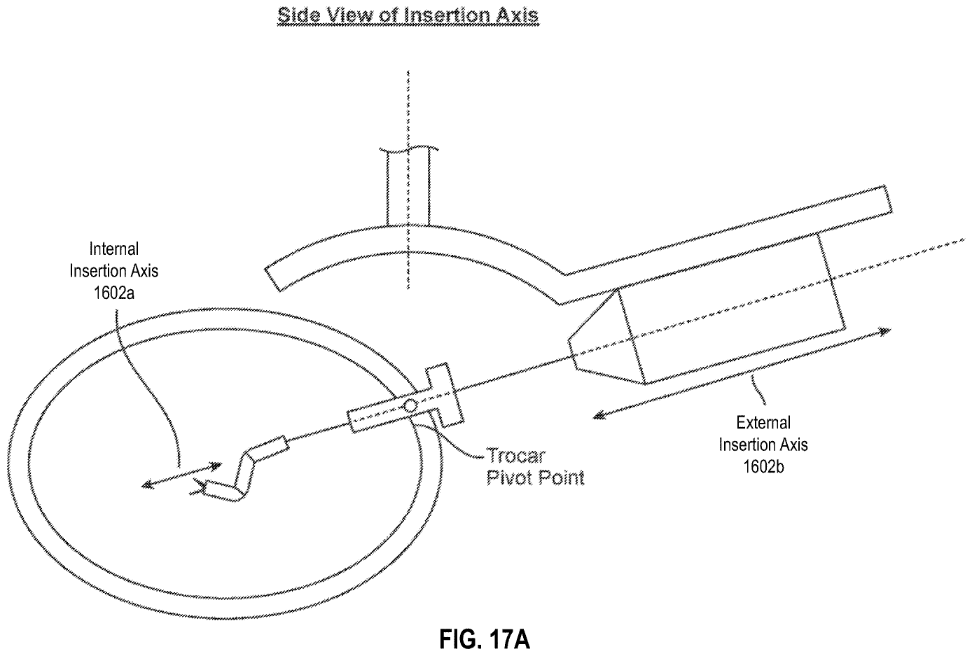

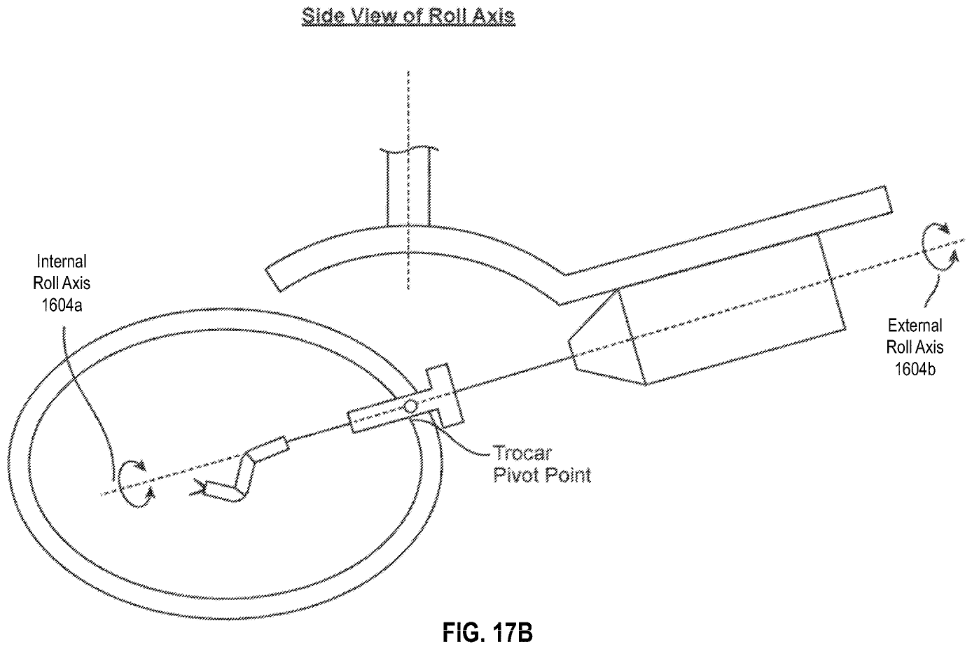

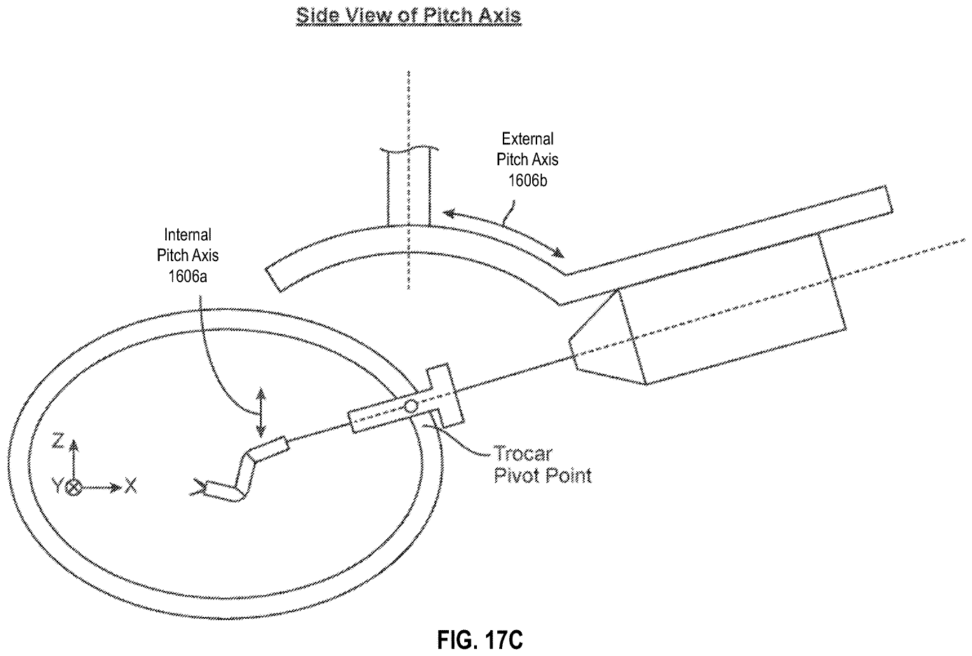

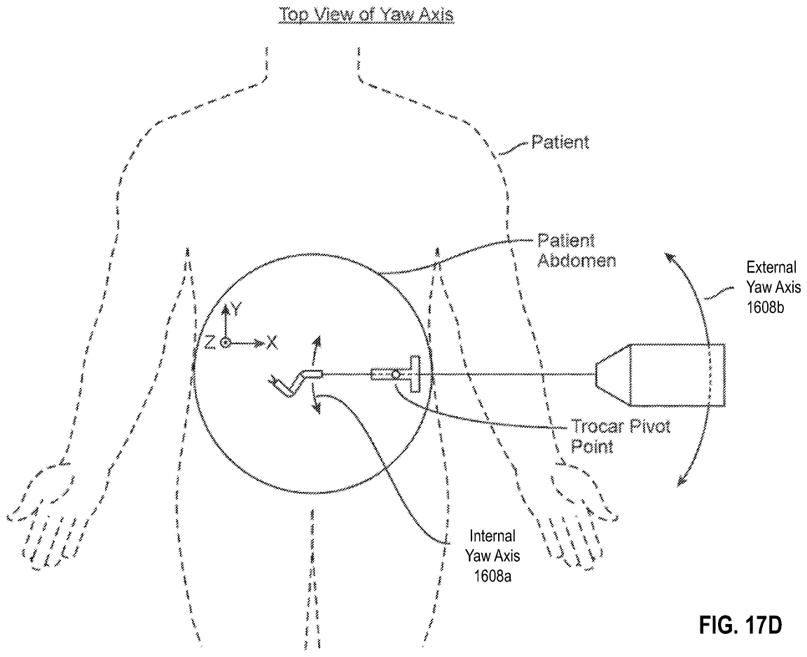

FIGS. 17A-17D show one example of various axes of the RSS 46, including a side view of an insertion axis (FIG. 17A), a side view of a roll axis (FIG. 17B), a side view of a pitch axis (FIG. 17C), and a top view of a yaw axis (FIG. 17D). FIG. 17A provides a side view of an insertion axis (e.g., the RSS insertion axis 1602), including an internal insertion axis 1602a (inside the body cavity of the subject) and an external insertion axis 1602b (outside the body cavity of the subject). FIG. 17B provides a side view of a roll axis (e.g., the RSS roll axis 1604), including an internal roll axis 1604a (inside the body cavity of the subject) and an external roll axis 1604b (outside the body cavity of the subject). FIG. 17C provides a side view of a pitch axis (e.g., the RSS pitch axis 1606), including an internal pitch axis 1606a (inside the body cavity of the subject) and an external pitch axis 1606b (outside the body cavity of the subject). FIG. 17D provides a top view of a yaw axis (e.g., the RSS yaw axis 1608), including an internal yaw axis 1608a (inside the body cavity of the subject) and an external yaw axis 1608b (outside the body cavity of the subject).

The RSS 46 can cause the positioning and orientation of the robotic arm to change by performing adjustments to one or more of the RSS roll axis 1604, the RSS pitch axis 1606, and/or the RSS yaw axis 1608. When the RSS roll axis 1604 and/or the RSS pitch axis 1606 are adjusted, the operating volume (e.g., the first volume 414 (see FIGS. 11D and 12C) and/or the second volume 424 (see FIGS. 11F and 12E)) is moved relative to the subject. More specifically, it may be desirable to change an orientation of the initial RSS insertion axis 46 in order to reposition the first volume 414 (see FIGS. 11D and 12C) and/or the second volume 424 (see FIGS. 11F and 12E) within the internal cavity 104 of the subject during an insertion process as described herein. The first volume 414 and/or the second volume 424 can be repositioned within the interior cavity 104 of the subject 100 relative to an initial RSS insertion axis 1602. For example, if sensitive tissue or an obstruction (e.g., an organ, a tumor, scar tissue, a foreign object, another surgical tool, and/or the like including combinations and/or multiples thereof) is encountered within the interior cavity 104 during an insertion process as taught herein, an operating volume of one or more of the robotic arms or the camera can be repositioned, for example, by repositioning of the first volume 414 and/or the second volume 424 via a change in the RSS roll axis 1604, the RSS pitch axis 1606, and/or the RSS yaw axis 1608.

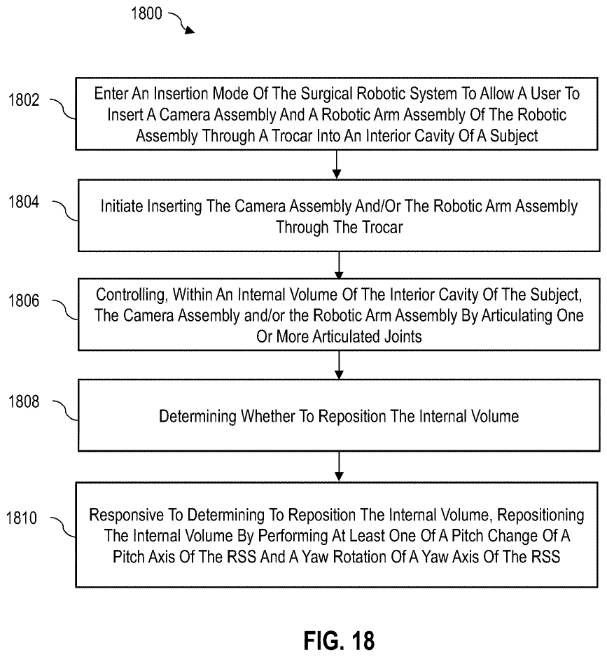

As an example, a pitch change along the RSS pitch axis 1606 and/or a yaw rotation along the RSS yaw axis 1608 can be performed on the RSS 46. The pitch change and/or the yaw rotation are performed relative to the insertion point (e.g., at a point where the trocar 50 enters the subject 100). The trocar 50, at the insertion point, acts as a pivot for the arms of the robotic arm assembly 42 for pitch and yaw movements. As a result, pitch and yaw movements of the support tube 122 of the robotic arm assembly 42 outside the subject 100 are realized in reverse within the internal cavity of the subject 100. More particularly, as the RSS 46 changes the initial RSS insertion axis 1602 by adjusting the external pitch axis 1606b and/or the external yaw axis 1608b, corresponding reverse movements occur within the cavity 104 of the subject 100 with respect to the internal pitch axis 1606a and/or the internal yaw axis 1608a. For example, if the RSS 46 causes the RSS insertion axis 1602 to pitch up outside the subject 100 by adjusting the external pitch axis 1606b, the camera assembly 44 as well as one or more arms of the robotic arm assembly 42 pitches down within the internal cavity 104 of the subject 100 because the pitch adjustment is realized by the internal pitch axis 1606a in reverse. Other movements are similar: for example, if the RSS 46 causes the RSS insertion axis 1602 to yaw right outside the subject 100 by adjusting the external yaw axis 1608b, the camera assembly 44 as well as one or more arms of the robotic arm assembly 42 yaws left within the internal cavity 104 of the subject 100 because the yaw adjustment is realized by the internal yaw axis 1608b in reverse. By providing for external movement (e.g., pitch rotation and/or yaw rotation) of the RSS 46, the operating volume within the internal cavity 104 of the subject 100 within which the camera assembly 44 and/or one or more of the end effectors 45 can be repositioned without an additional insertion point. The repositioning is described further with reference to FIG. 18.

FIG. 18 depicts a flowchart illustrating steps 1800 for repositioning a portion of the robotic assembly to avoid an obstruction in accordance with some embodiments.

In step 1802, the surgical robotic system 10 enters an insertion mode allowing a user to insert the camera assembly 44 and one or more robotic arms of the robotic arm assembly 42 of the robotic assembly 20 through the trocar 50 using the RSS 46 to position the camera assembly 44 and one or more robotic arms of the robotic arm assembly 42 into the interior cavity 104 of the subject 100. In some embodiments, a user can control the foot pedal array 19 to enter an insertion mode. In some embodiments, a user can control one of the hand controllers 201/202 and/or 261/262 to enter a menu mode, and the surgical robotic system 10 can display a menu on the display 12. The user can control the appropriate hand controller 201/202 and/or 261/262 to select an insertion mode on the menu. In some embodiments, the surgical robotic system 10 determines that the instrument tips 120 are installed properly (e.g., via data obtained from sensors associated with the instrument tips 120 or via a user input) and, in turn, the surgical robotic system 10 then automatically enters an insertion mode. It should be understood that a user can control one or both of hand controllers or one or both of the foot petals to enter an insertion mode.

At step 1804, the user via the surgical robotic system 10 initiates inserting the camera assembly 44 and/or one or more robotic arms 42 of the robotic arm assembly 42 through the trocar 50. Examples are described with respect to FIGS. 9A-9D. The RSS 46 performs the insertion along the RSS insertion axis 1602 as shown in FIG. 16.

At step 1806, the user via the surgical robotic system 10 controls, within an internal volume (e.g., the first volume 414 and/or the second volume 424) of the interior cavity 104 of the subject 100, the camera assembly 44 and/or one or more robotic arms 42 of the robotic arm assembly 42 by articulating one or more articulated joints as described herein. For example, one or more of the steps 308-322 can be performed at step 1806.

At step 1808, the surgical robotic system 10 determines whether to reposition the internal volume (e.g., the first volume 414 and/or the second volume 424). For example, the determining can include determining whether sensitive tissue or an obstruction (e.g., an organ, a tumor, scar tissue, a foreign object, another surgical tool, and/or the like including combinations and/or multiples thereof) is encountered within the interior cavity 104 of the subject 100. For example, an image captured by the camera assembly 44 can depict sensitive tissue or an obstruction (e.g., an organ, a tumor, scar tissue, a foreign object, another surgical tool, and/or the like including combinations and/or multiples thereof). The obstruction can be detected manually by the surgeon, automatically by the surgical robotic system 10 (e.g., using a trained machine learning model for detecting obstructions using images), and/or the like including combinations and/or multiples thereof.

At step 1810, if it is determined to reposition the first or second volume (e.g., an obstruction is detected and cannot be avoided solely by articulation of the one or more robotic arms, a hernia can be encountered and can be adjusted, and/or the like including combinations and/or multiples thereof), the first and/or second volume is repositioned by performing at least one of a pitch change of the RSS pitch axis 1606 of the RSS 46 and/or a yaw rotation of the RSS yaw axis of the RSS 46. By repositioning the first and/or second volume, the obstruction can be avoided or the impact of the obstruction can be reduced. For example, as described herein, the first volume 414 and/or the second volume 424 can be repositioned relative to the interior cavity 104 of the subject 100. For example, if an obstruction is encountered within the interior cavity 104 (at step 1808), the first and/or second volume can be repositioned by performing a pitch change and/or a yaw rotation of the RSS 46 as described herein, which causes the repositioning of the first volume 414 and/or the second volume 424. As an example, a yaw rotation and/or a pitch change relative to the insertion axis can be performed using the RSS as described with reference to FIGS. 17C and 17D. The yaw rotation and/or the pitch change are performed, respectively, along the RSS yaw axis 1608 and/or the RSS pitch axis 1606 about the insertion point (e.g., at a point where the trocar 50 enters the subject 100). The trocar 50, at the insertion point, therefore acts as a pivot for the RSS pitch axis 1606 and the RSS yaw axis 1608. As a result, movements of the RSS pitch axis 1606 and the RSS yaw axis 1608 outside the subject 100 are realized in reverse within the internal cavity of the subject 100. For example, if the RSS 46 causes the insertion axis 1602 to pitch up outside the subject 100 along the external pitch axis 1606b, the camera assembly 44 pitches down within the internal cavity 104 of the subject 100 along the internal pitch axis 1606a. Other movements are similar: for example, if the RSS 46 causes the insertion axis 1602 to yaw right outside the subject 100 along the external yaw axis 1608b, the camera assembly 44 yaws left within the internal cavity 104 of the subject 100 along the internal yaw axis 1608a. By providing for external movement (e.g., pitch rotation and/or yaw rotation) of the RSS 46, the volume within the internal cavity 104 of the subject 100 within which the camera assembly 44 and/or the end effector 45 can be repositioned without an additional insertion point.

According to one or more embodiments described herein, the RSS 46 (or portion thereof) can be repositioned to avoid the obstruction prior to and/or subsequent to selectively activating one or more articulated joints for articulation as described herein. For example, one or more of the steps 308 to 326 of FIG. 8, as described herein, can be performed.

It should be appreciated that the steps 1806, 1808, and 1810 can be performed iteratively. For example, it is possible that multiple obstacles can be encountered as the various articulated joints are enabled. For example, a first obstacle may be encountered in the first volume 414 during step 1806. The steps 1808 and 1810 can then be performed to reposition the first volume 414 to avoid the first obstacle. As additional articulated joints are enabled, and a second obstacle may be encountered in the second volume 424. In such cases, the steps 1808 and 1810 can be performed again to reposition the second volume 424 to avoid the second obstacle.

What is claimed is:

1. A surgical robotic system comprising:
a camera assembly;
a robotic arm assembly having a first robotic arm and a second robotic arm, each of the first and second robotic arms having a plurality of articulated joints;
hand controllers graspable by a user of the surgical robotic system to control the first and the second robot arms and the camera assembly;
a trocar;
a memory storing one or more instructions;
a processor configured to or programmed to read the one or more instructions stored in the memory, the processor operationally coupled to the robotic arm assembly, the hand controllers and the camera assembly to:
enter an insertion mode allowing the user to insert the camera assembly and robotic arm assembly through the trocar into an interior cavity of a subject;
determine that the first robotic arm is inserted in the trocar;
determine that a first articulated joint of the plurality of articulated joints of the first robotic arm exits the trocar and reaches a first articulated joint inserted position in the interior cavity, the first articulated joint inserted position indicating that the first articulated joint is free to rotate relative to the trocar;
allow a first hand controller of the hand controllers to articulate the first articulated joint within a first volume upon determination that the first articulated joint reaches the first articulated joint inserted position;
determine that a second articulated joint of the plurality of articulated joints of the first robotic arm exits the trocar and reaches a second articulated joint inserted position in the interior cavity, the second articulated joint inserted position indicating that the second articulated joint is free to rotate relative to the trocar; and
allow the first hand controller to articulate the second articulated joint within a second volume upon determination that the second articulated joint reaches the second articulated joint inserted position in the interior cavity.

2. The surgical robotic system of claim 1, wherein the processor is further configured to or programmed to read the one or more instructions stored in the memory to:
determine that the second robotic arm is inserted in the trocar;
determine that a first articulated joint of the second robotic arm exits the trocar and reaches a first articulated joint inserted position of the second robotic arm in the interior cavity, the first articulated joint inserted position of the second robotic arm indicating that the first articulated joint of the second robotic arm is free to rotate relative to the trocar; and
allow a second hand controller of the hand controllers to articulate the first articulated joint of the second robotic arm within a third volume upon determination that the first articulated joint of the second robotic arm reaches the first articulated joint inserted position of the second robotic arm.

3. The surgical robotic system of claim 2, wherein the processor is further configured to or programmed to read the one or more instructions stored in the memory to:
allow the user to insert the second robotic arm upon determination that the first robotic arm is fully inserted in the interior cavity.

4. The surgical robotic system of claim 2, wherein the processor is further configured to or programmed to read the one or more instructions stored in the memory to:
determine that the second robotic arm is fully inserted in the interior cavity; and
determine that the insertion process is complete.

5. The surgical robotic system of claim 4, wherein the surgical robotic system comprises a display, wherein the processor is further configured to or programmed to read the one or more instructions stored in the memory to:
operate the display to output one or more selectable menu items allowing the user to exit the insertion mode.

6. The surgical robotic system of claim 1, wherein the processor is further configured to or programmed to read the one or more instructions stored in the memory to:
determine that the first robotic arm is fully inserted in the interior cavity; and allow the first hand controller to fully articulate the first robotic arm.

7. The surgical robotic system of claim 1, wherein the processor is further configured to or programmed to read the one or more instructions stored in the memory to:
determine that the camera assembly is inserted through the trocar;
determine that the camera assembly exits the trocar and reaches a camera inserted position in the interior cavity;
allow the hand controllers to control a position and an orientation of the camera assembly in the interior cavity; and
operate the display to output an image captured by the camera assembly.

8. The surgical robotic system of claim 1, wherein the processor is further configured to or programmed to read the one or more instructions stored in the memory to:
operate the display to output a visual representation indicating that the first articulated joint or the second articulated joint is able to be articulated by the first hand controller to change a position and an orientation.

9. The surgical robotic system of claim 8, wherein the visual representation is color coded to represent which of the plurality of articulated joints is free to rotate within the interior cavity.

10. The surgical robotic system of claim 1, wherein the first robotic arm or the second robotic arm comprises a wrist hinge joint, an elbow hinge joint, a shoulder hinge joint, and an end-effector.

11. The surgical robotic system of claim 10, wherein the first volume is determined by a radius of a hand length and a height of a forearm length, wherein the hand length is a length between a tip of the end-effector and the wrist hinge joint, and the forearm length is a length between the wrist hinge joint and the elbow hinge joint.

12. The surgical robotic system of claim 11, wherein the second volume is determined by a radius of a sum of the forearm length and the hand length, and a height of an upper arm length, wherein the upper arm length is a length between the elbow hinge joint and the shoulder hinge joint.

13. A method for inserting a robotic assembly of a surgical robotic system through a trocar into an interior cavity of a subject, the robotic assembly including a robotic arm assembly, the method comprising:
inserting a first robotic arm of the robotic arm assembly through the trocar, the first robotic arm having a plurality of articulated joints;
determining that a first articulated joint of the plurality of articulated joints exits the trocar and reaches a first articulated joint inserted position in the interior cavity, the first articulated joint inserted position indicating that the first articulated joint is free to rotate relative to the trocar;
enabling, via a first hand controller of the surgical robotic system, articulation of the first articulated joint within a first volume in the interior cavity such that the first articulated joint is able to be articulated by the first hand controller within the first volume;
determining that a second articulated joint of the plurality of articulated joints exits the trocar and reaches a second articulated joint inserted position in the interior cavity, the second articulated joint inserted position indicating that the second articulated joint is free to rotate relative to the trocar; and
enabling, via the first hand controller, articulation of the second articulated joint within a second volume in the interior cavity such that the second articulated joint is able to be articulated by the first hand controller within the second volume.

14. The method of claim 13, further comprising:
inserting a second robotic arm of the robotic arm assembly through the trocar;
determining that a first articulated joint of the second robotic arm exits the trocar and reaches a first articulated joint inserted position of the second robotic arm in the interior cavity, the first articulated joint inserted position of the second robotic arm indicating that the first articulated joint of the second robotic arm is free to rotate relative to the trocar; and
enabling, via a second hand controller of the surgical robotic system, articulation of the first articulated joint of the second robotic arm within a first volume associated with the second robotic arm.

15. The method of claim 14, wherein inserting the second robotic arm occurs after the first robotic arm is fully inserted in the interior cavity.

16. The method of claim 14, further comprising:
determining that a second articulated joint of the second robotic arm exits the trocar and reaches a second articulated joint inserted position of the second robotic arm in the interior cavity, the second articulated joint inserted position of the second robotic arm indicating that the second articulated joint of the second robotic arm is free to rotate relative to the trocar;
enabling, via the second hand controller, articulation of the second articulated joint of the second robotic arm within a second volume associated with the second robotic arm;
determining that a third articulated joint of the second robotic arm exits the trocar and reaches a third articulated joint inserted position of the second robotic arm in the interior cavity, the third articulated joint inserted position of the second robotic arm indicating that the third articulated joint of the second robotic arm is free to rotate relative to the trocar;
determining that the second robotic arm is fully inserted in the interior cavity;
determining that the insertion process is complete; and
outputting one or more selectable menu items on a display allowing the user to exit the insertion mode.

17. The method of claim 13, further comprising:
determining that a third articulated joint of the plurality of articulated joints exits the trocar and reaches a third articulated joint inserted position in the interior cavity, the third articulated joint inserted position indicating that the third articulated joint is free to rotate relative to the trocar;
determining that the first robotic arm is fully inserted into the interior cavity; and
enabling, via the first hand controller, full articulation of the first robotic arm.

18. The method of claim 13, further comprising:
inserting a camera assembly of the robotic assembly through the trocar; and
controlling the camera assembly to reach a desired camera view.

19. The method of claim 13, further comprising:
outputting a visual representation of the first robotic arm on a display, the visual representation indicating that first articulated joint or the second articulated joint is able to be articulated by the first hand controller to change a position and an orientation.

20. The method of claim 19, wherein the visual representation is color coded to represent which of the plurality of articulated joints is free to rotate within the interior cavity.

21. The method of claim 13, wherein the first volume is less than the second volume.

22. The method of claim 13, wherein the first articulated joint is articulated by the first hand controller to manipulate tissue within the first volume.

23. The method of claim 13, wherein the first and second articulated joints are articulated by the first hand controller to manipulate tissue within the second volume.

24. The method of claim 14, wherein the first and second articulated joints of the first robotic arm and the first articulated joint of the second robotic arm are articulated by the first and second hand controllers to manipulate tissue within the interior cavity.

25. The method of claim 13, further comprising:
during the inserting of the first robotic arm of the robotic arm assembly through the trocar, detecting an obstruction within interior cavity the subject; and
repositioning at least a portion of the robotic assembly to avoid the obstruction.

26. The method of claim 25, wherein the repositioning comprises performing a yaw rotation of the robotic arm assembly, the yaw rotation being performed relative to an insertion axis.

27. The method of claim 25, wherein the repositioning comprises performing a pitch rotation of the robotic arm assembly, the pitch rotation being performed relative to an insertion axis.

28. A method for repositioning at least a portion of a robotic assembly of a surgical robotic system to avoid an obstruction, the method comprising:
entering an insertion mode of the surgical robotic system to allow a user to insert a camera assembly and a robotic arm assembly of the robotic assembly through a trocar into an interior cavity of a subject;
initiating inserting the camera assembly and the robotic arm assembly through the trocar;
controlling, within an internal volume of the interior cavity of the subject, the camera assembly and the robotic arm assembly by articulating one or more articulated joints;
determining whether to reposition the internal volume; and
responsive to determining to reposition the internal volume, repositioning the internal volume by performing at least one of a pitch change of a pitch axis of the robotic assembly and a yaw rotation of a paw axis of the robotic assembly.

29. The method of claim 28, wherein the yaw rotation is performed relative to an insertion axis.

30. The method of claim 28, wherein the pitch rotation is performed relative to an insertion axis.

31. The method of claim 28, wherein determining whether to reposition the internal volume comprises determining whether an obstruction is encountered within the interior cavity of the subject, wherein the repositioning is performed in order to avoid the obstruction.

32. The method of claim 28, wherein determining whether an obstruction is encountered is based at least in part on an image captured by the camera.
